(19) United States
(12) Patent Application Publication
(10) Pub. No.: US 2010/0273189 A1
(43) Pub. Date: Oct. 28, 2010

(54) NON SEPARATION ASSAYS WITH SELECTIVE SIGNAL INHIBITORS

(75) Inventors: Hashem AKHAVAN-TAFTI, Howell, MI (US); Renuka DE SILVA, Northville, MI (US); Terri MCLERNON, Mounds View, MN (US); James MENDOZA, Robbinsdale, MN (US); Michael SALVATI, Minnetrista, MN (US); Nir SHAPIR, Falcon Heights, MN (US); Wenhua XIE, Novi, MI (US)

Correspondence Address: Michael C. Schiffer - Legal Dept.;BECKMAN COULTER, INC., 250 S. Kraemer Boulevard, BREA, CA, 92821, US

(73) Assignee: BECKMAN COULTER, INC., Brea, CA (US)

(21) Appl. No.: 12/714,294

(22) Filed: Feb. 26, 2010

Related U.S. Application Data
(60) Provisional application No. 61/156,471, filed on Feb. 27, 2009.
(60) Provisional application No. 61/300,314, filed on Feb. 1, 2010.

(51) Int. Cl. G01N 33/53

(52) U.S. Cl. 435/7.9; 436/501

(57) ABSTRACT

Methods, reagents, kits and systems are disclosed for determining an analyte in a sample, the assay method comprising forming a reaction mixture in an aqueous solution, by adding a chemiluminescent-labeled immobilized specific binding member, an activator-labeled specific binding member, a selective signal inhibiting agent, and a sample, wherein the chemiluminescent-labeled immobilized specific binding member and activator-labeled specific binding member bind to analyte present in the sample to form a binding complex, and adding to the reaction mixture a trigger solution to release a detectable chemiluminescent signal correlated to the amount of the analyte-bound binding complex present in the reaction mixture.

### BACKGROUND

Specific binding assays are test methods for detecting the presence or amount of a substance and are based on the specific recognition and binding together of specific binding partners. Immunoassays are an example of a specific binding assay in which an antibody binds to a particular protein or compound. In this example an antibody is a member of a specific binding pair member. Nucleic acid binding assays are another type in which complementary nucleic acid strands are the specific binding pair. Specific binding assays constitute a broad and growing field of technology that enable the accurate detection of disease states, infectious organisms and drugs of abuse. Much work has been devoted over the past few decades to devise assays and assay methodology having the required sensitivity, dynamic range, robustness, broad applicability and suitability to automation. These methods can be grouped broadly into two categories.

Homogeneous methods utilize an analyte-specific binding reaction to modulate or create a detectable signal without requiring a separation step between analyte-specific and analyte non-specific reactants. Heterogeneous formats rely on physical separation of analyte-bound and free (not bound to analyte) detectably labeled specific binding partners. Separation typically requires that critical reactants be immobilized onto some type of solid substrate so that some type of physical process can be employed, e.g. filtration, settling, agglomeration or magnetic separation, and typically also require wash steps to remove the free detectably labeled specific binding partners.

Assay methods relying on producing a chemiluminescent signal and relating it to the amount of an analyte have experienced increasing use. Such methods can be performed with relatively simple instruments yet display good analytical characteristics. In particular, methods employing an enzyme-labeled specific binding partner for the analyte and a chemiluminescent enzyme substrate for detection have found widespread use. Common label enzymes include alkaline phosphatase and horseradish peroxidase.

U.S. Pat. No. 6,911,305 discloses a method of detecting polynucleotide analytes bound to a sensitizer or sensitizer-labeled probe on a first film. The film is contacted with a second film bearing an immobilized chemiluminescent precursor. Exciting the sensitizer in the sandwiched films produces singlet oxygen which reacts with the chemiluminescent precursor to produce a triggerable chemiluminescent compound on the second film. The triggerable chemiluminescent compound is reacted with a reagent to generate chemiluminescence on the second film for detecting the analyte. These methods do not rely on the specific binding reaction for bringing the reactants into contact; rather the second film serves as a reagent delivery device.

U.S. Pat. No. 6,406,913 discloses assay methods comprising treating a medium suspected of containing an analyte under conditions such that the analyte causes a photosensitizer and a chemiluminescent compound to come into close proximity. The photosensitizer generates singlet oxygen when irradiated with a light source; the singlet oxygen diffuses through a solution to and activates the chemiluminescent compound when it is in close proximity. The activated chemiluminescent compound subsequently produces light. The amount of light produced is related to the amount of analyte in the medium. In one embodiment, at least one of the photosensitizer or the chemiluminescent compound is associated with a suspendable particle, and a specific binding pair member is bound thereto,

U.S. patent application publications US20070264664 and US20070264665 disclose assay methodology for performing specific binding pair assays involving reaction of immobilized chemiluminescent compounds with activator compounds brought into a reactive configuration by virtue of the specific binding reaction. No separation or removal of the excess unbound chemiluminescent compound or activator is required. These assay formats provide superior operational convenience and flexibility in automation compared to prior art assay techniques. Despite these advantages, additional improvements in assay design and performance remain a goal of assay developers. The assay methods of the present disclosure address these needs by providing simple assay methods of improved sensitivity.

### DEFINITIONS

Alkyl—A branched, straight chain or cyclic hydrocarbon group containing from 1-20 carbons which can be substituted with 1 or more substituents other than H. Lower alkyl as used herein refers to those alkyl groups containing up to 8 carbons.

Analyte—A substance in a sample to be detected in an assay. One or more substances having a specific binding affinity to the analyte will be used to detect the analyte. The analyte can be a protein, a peptide, an antibody, or a hapten to which an antibody that binds it can be made. The analyte can be a nucleic acid or oligonucleotide which is bound by a complementary nucleic acid or oligonucleotide. The analyte can be any other substance which forms a member of a specific binding pair. Other exemplary types of analytes include drugs such as steroids, hormones, proteins, glycoproteins, mucoproteins, nucleoproteins, phosphoproteins, drugs of abuse, vitamins, antibacterials, antifungals, antivirals, purines, antineoplastic agents, amphetamines, azepine compounds, nucleotides, and prostaglandins, as well as metabolites of any of these drugs, pesticides and metabolites of pesticides, and receptors. Analyte also includes cells, viruses, bacteria and fungi.

Activator—a compound, also may be referred to as a label, that effects the activation of the chemiluminescent compound so that, in the presence of a trigger, chemiluminescence is produced.

Activator-labeled sbm or activator-specific binding member conjugate—a reactant in the assay mix that includes at least the following in a connected configuration: a) a specific binding member for an analyte and b) an activator compound or label that effects activation of a chemiluminescent compound.

Antibody—includes the native and engineered full immunoglobulin as well as native and engineered portions and fragments thereof.

Aralkyl—An alkyl group substituted with an aryl group. Examples include benzyl, benzyhydryl, trityl, and phenylethyl.

Aryl—An aromatic ring-containing group containing 1 to 5 carbocyclic aromatic rings, which can be substituted with 1 or more substituents other than H.

Biological material—includes, for example. whole blood, anticoagulated whole blood, plasma, serum, tissue, animal and plant cells, cellular content, viruses, and fungi.

Chemiluminescent compound—A compound, which also may be referred to as a label, which undergoes a reaction so as to cause the emission of light, for example by being converted into another compound formed in an electronically excited state. The excited state may be either a singlet or triplet excited state. The excited state may directly emit light upon relaxation to the ground state or may transfer excitation energy to an emissive energy acceptor, thereby returning to the ground state. The energy acceptor is raised to an excited state in the process and emits light.

Chemiluminescent-labeled immobilized sbm—a reactant in the assay mix that includes at least the following in a connected configuration: a) a specific binding member for an analyte, b) a chemiluminescent compound or label, and c) a solid phase.

Connected—as used herein indicates that two or more chemical species or support materials are chemically linked, e.g. by one or more covalent bonds, or are passively attached, e.g. by adsorption, ionic attraction, or a specific binding process such as affinity binding. When such species or materials are connected with each other, more than one type of connection can be involved.

Heteroalkyl—An alkyl group in which at least one of the ring or non-terminal chain carbon atoms is replaced with a heteroatom selected from N, O, or S.

Heteroaryl—An aryl group in which one to three of the ring carbon atoms is replaced with a heteroatom selected from N, O, or S. Exemplary groups include pyridyl, pyrrolyl, thienyl, furyl, quinolyl and acridinyl groups.

Magnetic particles—As used herein encompasses particulate material having a magnetically responsive component. Magnetically responsive includes ferromagnetic, paramagnetic and superparamagnetic materials. One exemplary magnetically responsive material is magnetite. Particles can have a solid core portion that is magnetically responsive and is surrounded by one or more non-magnetically responsive layers. Alternately the magnetically responsive portion can be a layer around or can be particles disposed within a non-magnetically responsive core.

Sample—A mixture containing or suspected of containing an analyte to be measured in an assay. Analytes include for example proteins, peptides, nucleic acids, hormones, antibodies, drugs, and steroids Typical samples which can be used in the methods of the disclosure include bodily fluids such as blood, which can be anticoagulated blood as is commonly found in collected blood specimens, plasma, serum, urine, semen, saliva, cell cultures, tissue extracts and the like. Other types of samples include solvents, seawater, industrial water samples, food samples and environmental samples such as soil or water, plant materials, eukaryotes, bacteria, plasmids, viruses, fungi, and cells originated from prokaryotes.

SSIA, (Selective Signal Inhibiting Agent)—A compound provided in an assay reaction mixture of the present disclosure such that non-specific signal or background signal is reduced in a greater amount than the analyte-specific signal generated from the chemiluminescent production reaction of the assay reaction mixture.

Solid support—a material having a surface upon which assay components are immobilized. Materials can be in the form of particles, microparticles, nanoparticles, metal colloids, fibers, sheets, beads, membranes, filters and other supports such as test tubes, microwells, chips, glass slides, and microarrays.

Soluble, solubility, solubilize—The ability or tendency of one substance to blend uniformly with another. In the present disclosure, solubility and related terms generally refer to the property of a solid in a liquid, for example SSIA in an aqueous buffer. Solids are soluble to the extent they lose their crystalline form and become molecularly or ionically dissolved or dispersed in the solvent (e.g. liquid) to form a true solution. In contrast: two-phase systems where one phase consists of small particles (including microparticles or colloidal sized particles) distributed throughout a bulk substance, whether stabilized to deter precipitation or unstabilized.

Substituted—Refers to the replacement of at least one hydrogen atom on a group by a non-hydrogen group. It should be noted that in references to substituted groups it is intended that multiple points of substitution can be present unless clearly indicated otherwise.

Test device—A vessel or apparatus for containing the sample and other components of an assay according to the present invention. Included are, for example, test tubes of various sizes and shapes, microwell plates, chips and slides on which arrays are formed or printed, test strips and membranes.

### IN THE DRAWINGS

FIG. 1A is a plot demonstrating the influence of pH on background chemiluminescence in a chemiluminescent reaction of the present methods as described in Example 10.

FIG. 1B is a plot demonstrating the influence of pH on specific signal chemiluminescence in a chemiluminescent reaction of the present methods as described in Example 10.

FIG. 2A is a plot illustrating the detection of cTnI in an immunoassay method as described in Example 17.

FIG. 2B is a plot illustrating the detection of cTnI in a dilution series in an immunoassay method as described in Example 17.

FIG. 2C is a plot illustrating the detection of cTnI in a dilution series in an immunoassay method as described in Example 17.

FIG. 3 is a plot illustrating a comparison of the results of a cTnI assay conducted by the methods of the present invention compared with the results of a reference method as described in Example 18.

### DESCRIPTION OF THE INVENTION

The present disclosure provides improved assay methodology for determining an analyte in a sample. In particular, this disclosure describes analyte-specific binding assays which do not require a separation step and provide improvement in analyte specific-signal response over non-specific signal or background.

Surprisingly, Applicants have discovered that such assay methods can be further improved by the use of a selective signal inhibiting agent, SSIA. In the present methods, addition of the SSIA to assay systems where excess activator and/or excess chemiluminescent compound is not removed markedly improves the ability to perform sensitive, specific, analyte-concentration dependent binding assays. Assay precision and sensitivity are thereby improved, leading to more reliable and useful tests. This improvement was not expected or predictable. By use of the SSIA, the ratio of signal produced by reaction between immobile chemiluminescent label and activator label, both associated in a complex of labeled specific binding pair members with an analyte, to signal from the labels present but not in such a complex is dramatically improved. In addition, background effects at low, levels of analyte are minimized.

The methods previously disclosed in U.S. patent application publications US20070264664 and US20070264665 provided improved, rapid and simple assay methods for detecting the presence, location, or amount of substances by use of analyte-specific binding reactions. The assay methods involve reaction of immobilized chemiluminescent compounds with activator compounds brought into a reactive configuration by virtue of an analyte-mediated specific binding reaction. Assays and methods are performed without separating free specific binding partners from specific binding partners bound in complexes.

The present methods require the use of an immobilized analyte specific binding member connected with a chemiluminescent label, a non-immobilized analyte specific binding member for an analyte connected with an activator for reaction with the chemiluminescent label, and a selective signal inhibiting agent. Addition of a trigger solution initiates the emission of chemiluminescence for detecting the analyte. Assays and methods are performed without separating free specific binding partners from specific binding partners bound in complexes.

The present disclosure is concerned with improved, rapid, and simple assay methods for detecting the presence, location, or amount of substances by means of analyte-specific binding reactions. The methods require the use of an immobilized analyte specific binding member and a non-immobilized analyte specific binding member for an analyte. One analyte specific binding member is associated with a chemiluminescent label, while the other analyte specific binding member is associated with an activator. In many embodiments, the activator compound, which induces a chemiluminescent reaction, is brought in proximity with the chemiluminescent label on a solid support, as mediated by either or both analyte specific binding members binding with analyte, in aqueous solution containing analyte, enhancer, selective signal inhibiting agent and a trigger solution, thereby generating a detectable chemiluminescent signal related to analyte concentration. In other embodiments, on a solid support, the activator compound, which induces a chemiluminescent reaction, is blocked from being in proximity with the chemiluminescent label, as mediated by one analyte specific binding member competing with analyte for binding to the other analyte-specific binding member, in aqueous solution containing analyte, enhancer, selective signal inhibiting agent and a trigger solution, thereby generating a detectable chemiluminescent signal inversely related to analyte concentration.

In many embodiments, one analyte specific binding member (“sbm”) is connected with a solid support and a chemiluminescent label (“chemiluminescent-labeled immobile sbm”), while another analyte specific binding member is connected with an activator (“activator-labeled sbm”) that is non-immobilized in aqueous solution. The chemiluminescent-labeled immobile sbm, activator-labeled sbm, enhancer, selective signal inhibiting agent, sample and a trigger solution produce detectable signal when the activator is brought into operable proximity to the immobilized chemiluminescent compound so that it is effective to activate a reaction generating chemiluminescence upon addition of a trigger solution. By operable proximity is meant that the chemiluminescent compound and activator are close enough, including and up to physical contact, that they can react. In many embodiments, activator-labeled specific binding member is provided to the system in excess to the amount needed to determine analyte presence, location or concentration.

In one aspect, the present methods differ from most conventional test methods in that the chemiluminescent compound and the activator are both spatially constrained via analyte specific binding reaction of one or more analyte specific binding members in operable proximity at a solid support to permit a chemiluminescent reaction to be performed upon addition of a trigger solution. Commonly owned patent application PCT WO 2007/013398 teaches assay methods in which the presence of excess non-immobilized or immobilized member, if not removed, does not defeat the ability to perform sensitive specific binding assays. For example, non-immobilized activator is not removed prior to addition of trigger solution and detection since its presence does not prevent the chemiluminescent detection signal from being correlated with the amount of the analyte.

The function of the SSIA in improving assay sensitivity is understood in reference to Scheme 1. Combinations of free and complexed chemiluminescent-labeled sbm and activator-labeled sbm can contribute to the observed chemiluminescent signal when trigger solution is added.

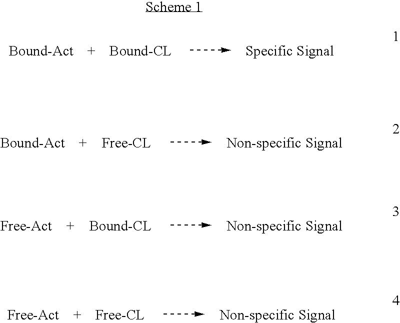

As shown in the scheme, reaction 1 produces a signal that is relatable to the amount of analyte in an assay. The SSIA achieves its surprising function, at least in part, by selectively inhibiting or depressing the amount of signal from reaction 2 in relation to that from reaction 1. The SSIA may also improve signal:background ratio by suppressing signal generation from exogenous interfering substances.

In one embodiment there are provided assay methods, in particular binding assay methods, in which an chemiluminescent-labeled immobile sbm compound, an activator-labeled sbm, are brought into operable proximity via at least one specific binding reaction due to the presence of an analyte, wherein the bound activator conjugate activates a reaction generating chemiluminescence in the presence of selective signal inhibiting agent and enhancer upon addition of a trigger solution for detecting the presence, location or amount of the analyte.

In some other embodiments, a competitive assay format is utilized where an activator-labeled sbm competes with analyte for binding with chemiluminescent-labeled immobile sbm, thereby generating chemiluminescence in an inverse relationship with analyte concentration or competition assay. In such embodiments, activator is brought into operable proximity to the immobilized chemiluminescent compound by activator-labeled sbm binding to chemiluminescent-labeled immobile sbm to activate a reaction generating chemiluminescence upon addition of a trigger solution in the presence of enhancer. Chemiluminescent signal decreases as analyte concentration increases thereby competitively blocking binding of activator-labeled sbm binding to chemiluminescent-labeled immobile sbm.

The assay components, such as: sample containing analyte, activator-labeled sbm, chemiluminescent-labeled immobile sbm, selective signal inhibiting agent and enhancer can be added in various orders and combinations to a test vessel, without washing or separations, and the luminescence read upon addition of trigger solution. In one embodiment, for example, sample and activator-labeled sbm and/or chemiluminescent-labeled immobile sbm can be pre-mixed. In one embodiment, SSIA can be included in a premix with activator-labeled sbm and/or chemiluminescent-labeled immobile sbm and/or sample. Enhancer can be included in a premix or added with the trigger solution. No washing or separation of excess unbound reactants is required.

Conventional assays using chemiluminescent substrates and enzyme labeled conjugates provide the chemiluminescent substrate in great excess to the amount of label enzyme. Frequently, the molar ratio of substrate/enzyme can exceed nine powers of ten, i.e., a billion-fold excess. It is believed to be necessary in conventional assays to supply such an enormous excess of chemiluminescent compound in order to ensure an adequate supply of substrate for continuous enzymatic turnover and that this process guarantees adequate detection sensitivity in assay methods. Applicants have found that it is possible to devise highly sensitive assay methods that reduce the ratio of chemiluminescent compound to activator by several orders of magnitude. In this regard these methods described herein differ fundamentally from known enzyme-linked assay methods.

Eliminating washing and separation steps as described above and as demonstrated in exemplary assays described below affords opportunities to simplify the design of assay protocols. The reduced number of operational steps decreases assay time, inter-assay variability from incomplete washing, and cost. At the same time it enhances the ability to automate and miniaturize assay performance with all of the of the inherent advantages attendant on automation and miniaturization.

Generally, assays performed according to the present methods, a solid support is provided in a test device for specifically capturing an analyte of interest. The solid support is provided with an immobilized specific binding member for directly or indirectly binding an analyte to be detected. The solid support is further provided with a label, in many embodiments a chemiluminescent label, immobilized thereon.

An activator-labeled sbm is also introduced to the test device. The activator-labeled sbm and chemiluminescent-labeled immobile sbm are permitted to form specific binding complexes in the presence of a sample containing analyte. The sample, activator-labeled sbm, chemiluminescent-labeled immobile sbm, SSIA, and enhancer can be added separately in any order, or simultaneously, or can be pre-mixed and added as a combination. Time periods to allow binding reactions to occur (“incubations”) can be inserted at between or after any addition prior to triggering the reaction.

Finally, trigger solution is added to produce the chemiluminescence for detecting the analyte and the chemiluminescence is detected. Trigger solution minimally contains a peroxide as described further below, but may also contain enhancer and sometimes SSIA. Typically either peak light intensity level, total RLU's over a designated time period or total integrated light intensity is measured. The quantity of light can be related to the amount of the analyte by constructing a calibration curve according to generally known methods. When light emission ensues rapidly upon addition of trigger solution it is desirable to either mechanically time the onset of measurement to the addition by use of a suitable injector or to perform the addition with the test device already exposed to the detector. Optimum quantities of reactants, volumes, dilutions, incubation times for specific binding pair reactions, concentration of reactants, etc., can be readily determined by routine experimentation, by reference to standard treatises on methods of performing specific binding assays and using as a guide the specific examples described in detail below.

The concentration or amount of the analyte-specific binding members used in the present methods and assays will depend on such factors as analyte concentration, the desired speed of binding/assay time, cost and availability of conjugates, the degree of nonspecific binding of analyte-specific binding members. Usually, the analyte-specific binding members will be present in at least equal to the minimum anticipated analyte concentration, more usually at least the highest analyte concentration expected, and for noncompetitive assays the concentrations may be 10-10⁶ times the highest analyte concentration but usually less than 10⁻⁴ M, preferably less than 10⁻⁶ M, frequently between 10⁻¹¹ and 10⁻⁷ M. The amount of activator or chemiluminescent compound connected with a sbm member will usually be at least one molecule per analyte-specific binding members and may be as high as 10⁵, usually at least 10-10⁴ when the activator or chemiluminescent molecule is immobilized on a particle. Exemplary ratios of activator to chemiluminescent compound are provided in the worked examples.

Selective Signal Inhibiting Agents (SSIA)

The selective signal inhibiting agents of the present invention are compounds that when included in an assay reaction mixture comprising free and/or analyte-bound chemiluminescent-labeled sbm, free and/or analyte-bound activator-labeled sbm, enhancer and a trigger solution, such that the resulting signal from the analyte-bound labeled sbm members exceed background signal by a significantly greater degree than occurs in the absence of the SSIA.

One or more selective signal inhibiting agents are present in reaction methods at concentration between 10⁻⁶ M and 10⁻¹ M, frequently between 10⁻⁶ M and 10⁻² M, often between 10⁻⁵ M and 10⁻³ M, sometimes between 10⁻⁵ M and 10⁻⁴ M. In some embodiments, a selective signal inhibiting agent is present between 5×10⁻⁶M and 5×10⁻⁴ M in reactions according to the present methods. In still further embodiments, a selective signal inhibiting agent is present between 5×10⁻⁵ M and 5×10⁻⁴ M in reactions according to the present methods.

The selective signal inhibiting agent can be supplied as a separate reagent or solution at a higher concentration than is intended in the reaction solution. In this embodiment a measured amount of the working solution is dosed into the reaction solution to achieve the desired reaction concentration. In another embodiment the selective signal inhibiting agent is combined into a solution containing one or more of the labeled sbm members. In another embodiment the selective signal inhibiting agent is provided as a component of the trigger solution.

The degree to which the selective signal inhibiting agent improves the signal:background ratio will vary depending on the identity of the compound and the concentration at which it is used, among other factors. The degree can be framed in terms of an improvement factor in which the signal:background ratio of an assay at a particular analyte concentration wherein the assay is performed with the selective signal inhibiting agent is compared to the signal:background ratio of an assay at the same analyte concentration without the selective signal inhibiting agent. An improvement factor >1 is a gauge of an improved assay and evidence of a beneficial effect of the selective signal inhibiting agent. In embodiments of the invention improvement factors of at least 2, such as at least 5 and including at least 10, or at least 50 are achieved. It will be seen in reference to the examples below, that improvement factors can vary within an assay as a function of the analyte concentration. For example, improvement factors may increase as analyte concentration increases. In another embodiment the variation in improvement factor across a concentration may result in a more linear calibration curve, i.e. plot of chemiluminescence intensity vs. analyte concentration.

The following table lists, without limitation, compounds capable of functioning effectively as selective signal inhibiting agents. Additional compounds, not explicitly recited, can be found using the teachings of the present disclosure, including by routine application of the assay and screening test methods described in the examples.

TABLE 1
SELECTIVE SIGNAL INHIBITING AGENTS

| Glutathione | Ascorbic acid, including | |
|---|---|---|
| | ascorbate anion and salts thereof | |
| | 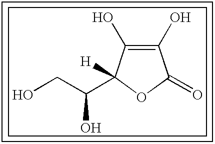 | |
| Uric Acid | L-Ascorbic acid 6-Palmitate | |
| (±)-a-Tocopherol | 5,6-Isopropylidene-L-Ascorbic acid | |
| (+)-y-Tocopherol | Butylated Hydroxytoluene (BHT) | |
| 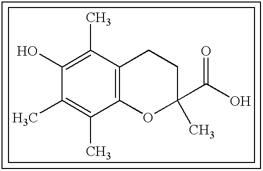 | 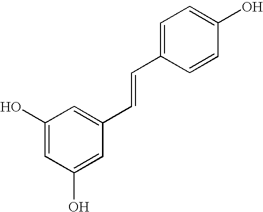 | |
| 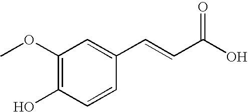 | 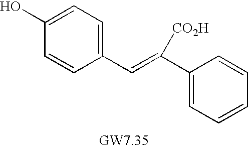 | |
| 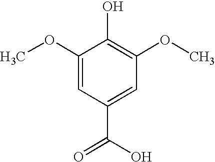 | 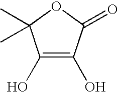 | |
| 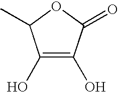 | 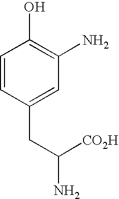 | |
| 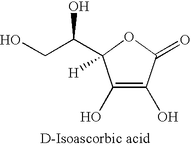 | 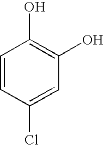 | |
| 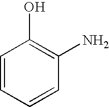 | 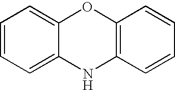 | |
| 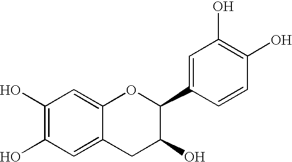 | Na₂SO₃ | Et₂NOH |

In some embodiments the selective signal inhibiting agent is selected from dialkylhydroxylamines. In some embodiments the selective signal inhibiting agent is selected from aromatic compounds having at least two hydroxyl groups oriented in an ortho-, or para-relationship. Exemplary compounds include:

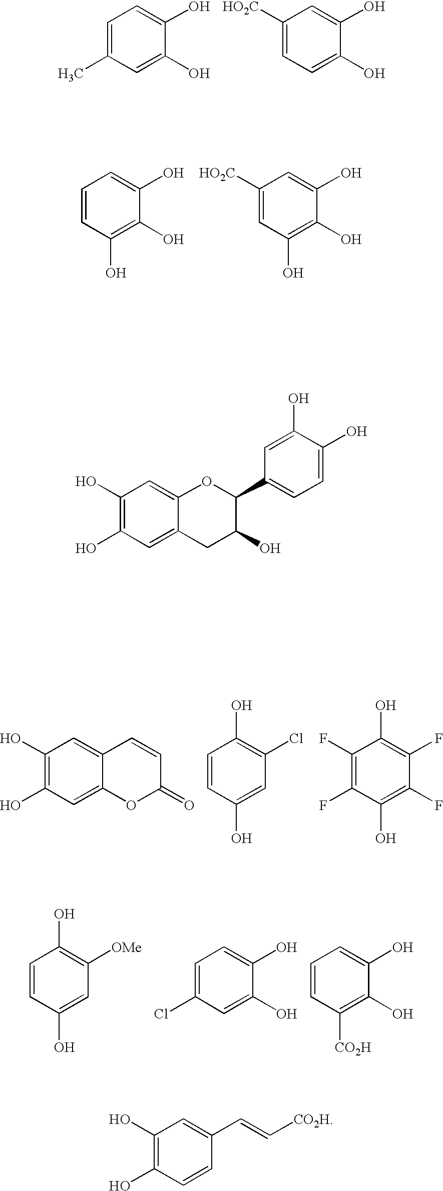

In some other embodiments the selective signal inhibiting agent is selected from aromatic compounds having at least a hydroxyl group and an amino group oriented in an ortho-, or para-relationship. Exemplary compounds include:

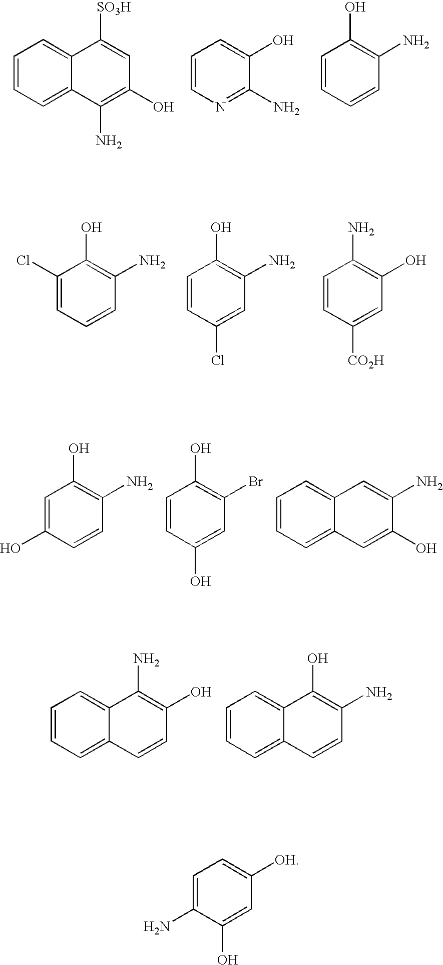

In yet other embodiments the selective signal inhibiting agent is selected from compounds having at least two hydroxyl groups substituted on a C—C double bond, also known as an enediol. Exemplary compounds include:

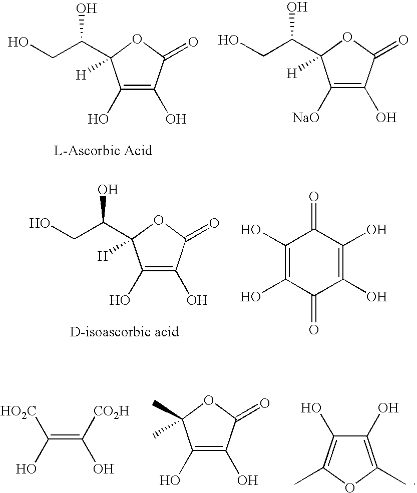

In one embodiment the selective signal inhibiting agent is selected from nitrogen heterocyclic compounds. Exemplary compounds include:

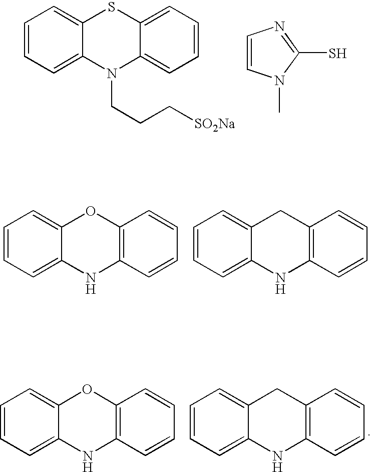

In one embodiment the selective signal inhibiting agent is supplied in masked form as a compound that is convertible into the active SSIA upon contact with peroxide. Suitable masked SSIA compounds are for example selected from hydroxyl- or amino-substituted arylboronic acid compounds. Exemplary compounds include:

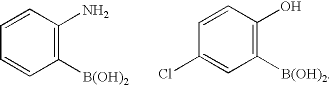

In one embodiment the selective signal inhibiting agent is selected from

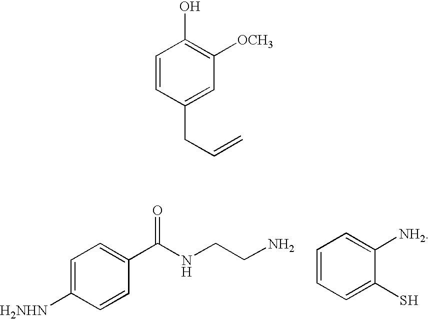

In various embodiments, one or more of the above selective signal inhibiting agents are used in combination in assay methods, assays or kits of the present disclosure.

In some embodiments, selective signal inhibiting agents have solubility in aqueous solution at 10 times working solution. Working solution is defined as a concentrated aqueous solution, such that a portion of the concentrated solution is added to the reaction mix to give the final concentration required after the addition of trigger solution.

Suitable aqueous solutions for working solutions of selective signal inhibiting agent include one or more of the following additional components: salts, biological buffers, alcohols, including ethanol, methanol, glycols, and detergents. In some embodiments, aqueous solutions include Tris buffered aqueous solutions, such as Buffer II (TRIS buffered saline, surfactant, <0.1% sodium azide, and 0.1% ProClin 300 (Rohm and Haas) available commercially from Beckman Coulter, Inc., Brea Calif.), 25% Ethanol/75% Buffer II, 25% Ethanol/75% Triton-X-100 (1%), or 10% 0.1 N NaOH/90% Buffer II.

Solid Phase Supports

In many embodiments the methods of the present disclosure, the chemiluminescent label is immobilized to a component of the test system. The label may be provided in a number of different ways as described in more detail below. In each variant the label is stably or irreversibly attached to a substance or material in a way that renders it immobile. By “irreversibly” it is intended that the label is not substantially removed from the solid support under the conditions of use in the intended assay. Passive or noncovalent attachment is also contemplated provided that the label is stably attached and retained on the solid support under the conditions of use. This can be accomplished in any of several ways.

In embodiments of the present disclosure for performing an assay, the chemiluminescent label becomes immobilized to a surface of a solid support. The analyte is attracted to the surface of the solid support, e.g., by an unlabeled analyte-specific binding member. The chemiluminescent label is brought into a reactive configuration with the activator by virtue of a specific binding reaction bringing the activator near the immobilized chemiluminescent label attached to the solid support. Then the trigger solution is added and chemiluminescence measured.

In one embodiment the chemiluminescent label is covalently linked to an immobilized analyte-specific binding member. An example would be a labeled capture antibody or antibody fragment immobilized on the wells of a microplate or on a particle. Immobilization of the analyte-specific binding member can be by covalent linkage or by an adsorption process. In this format, the chemiluminescent label is brought into a reactive configuration with the activator by virtue of two specific binding partners both binding an analyte in a “sandwich” format.

In another embodiment, the chemiluminescent label is covalently linked to an auxiliary substance that is immobilized on the solid support in a random manner. Immobilization of the auxiliary substance can be by covalent linkage or by an adsorption process. The label is thereby distributed more or less uniformly about the surface of the solid support. The analyte is attracted to the surface of the solid support, e.g., by an unlabeled analyte-specific binding member. The chemiluminescent label is brought into a reactive configuration with the activator by virtue of a specific binding reaction bringing the activator near the chemiluminescent label attached to the auxiliary substance attached or passively coated onto the surface of the support.

In another embodiment the chemiluminescent label is covalently linked to an immobilized universal antibody that has binding affinity for an analyte specific capture antibody.

In another embodiment the auxiliary substance to which the chemiluminescent label is covalently linked is a protein or peptide. Exemplary proteins include albumin or streptavidin (SA). The chemiluminescent compound can be provided for immobilization by using a biotin-chemiluminescent compound conjugate. Assay formats of this type can provide the analyte-specific binding member as a biotin conjugate, or by direct immobilization to the solid support or by indirect attachment through a universal capture component such as a species specific anti-immunoglobulin.

In another embodiment the auxiliary substance to which the chemiluminescent label is covalently linked is a synthetic polymer. Assay formats using polymeric auxiliaries for immobilizing the chemiluminescent compound can provide the analyte-specific binding member as a biotin conjugate, or by direct immobilization to the solid support or by indirect attachment through a universal capture component such as a species specific immunoglobulin.

In another embodiment, the chemiluminescent label is covalently linked to the surface of the solid support. In such an embodiment, the label is thereby distributed more or less uniformly about the surface of the solid support. The analyte is attracted to the surface of the solid support, e.g., by an unlabeled analyte-specific binding member. The chemiluminescent label is brought into a reactive configuration with the activator by virtue of a specific binding reaction bringing the activator near the chemiluminescent label directly attached to the surface of the support. Then, without washing or separation, the trigger solution is added and chemiluminescence measured.

In another embodiment an analog of the analyte is used comprising an activator-analyte analog conjugate. In another embodiment a labeled analyte is used comprising an activator-analyte conjugate. The activator-analyte analog conjugate or activator-analyte conjugate and analyte will competitively bind with the analyte-specific binding member. It will be apparent that in this type of assay method a negative correlation between the amount of analyte in the sample and the intensity of chemiluminescence will result.

In addition to attachment of chemiluminescent label through antibodies for binding antigens or other proteins or other antibodies via an immunoassay, the present methods can use chemiluminescent-labeled nucleic acids for detecting nucleic acids through binding of complementary nucleic acids. The use in this regard is not particularly limited with regard to the size of the nucleic acid, the only criterion being that the complementary partners be of sufficient length to permit stable hybridization. Nucleic acids as used herein include gene length nucleic acids, shorter fragments of nucleic acids, polynucleotides and oligonucleotides, any of which can be single or double stranded. In the practice of the disclosure using nucleic acids as analyte-specific binding members, a nucleic acid is covalently attached or physically immobilized on a surface of a solid support to capture an analyte nucleic acid. The chemiluminescent label can be attached to the capture nucleic acid or the label can be connected with the support as explained above. The capture nucleic acid will have full or substantially full sequence complementarity to a sequence region of the analyte nucleic acid.

When substantially complementary, the capture nucleic acid may possess a terminal overhanging portion, a terminal loop portion or an internal loop portion that is not complementary to the analyte provided that it does not interfere with or prevent hybridization with the analyte. The reverse situation may also occur where the overhang or loop resides within the analyte nucleic acid. Capture nucleic acid, analyte nucleic acid, a conjugate of an activator, and a third nucleic acid are allowed to hybridize. The third nucleic acid is substantially complementary to a sequence region of the analyte nucleic acid different from the region complementary to the capture nucleic acid. The hybridization of the capture nucleic acid and activator conjugate nucleic acid with the analyte can be performed consecutively in either order or simultaneously. As a result of this process, the chemiluminescent label becomes associated with the activator by virtue of specific hybridization reactions bringing the activator near the chemiluminescent label attached to the surface of the support. Trigger solution is provided and chemiluminescence detected as described above.

Another embodiment comprises a variation wherein a conjugate of the analyte with the activator is used. The analyte nucleic acid-activator conjugate and analyte nucleic acid will competitively bind with the analyte-specific binding member. It will be apparent that in this type of assay method a negative correlation between the amount of analyte in the sample and the intensity of chemiluminescence will result.

In addition to antibody-based and nucleic acid-based systems, other specific binding pairs as are generally known to one of ordinary skill in the art of binding assays can serve as the basis for test methods according to the present disclosure. Antibody-hapten pairs can also be used. Fluorescein/anti-fluorescein, digoxigenin/anti-digoxigenin, and nitrophenyl/anti-nitrophenyl pairs are exemplary. As a further example, the well known (strept)avidin/biotin binding pair can be utilized. To illustrate one way in which this binding pair could be used a streptavidin-chemiluminescent label conjugate can be covalently linked or adsorbed onto a solid support. A biotin-labeled analyte and an activator conjugate is then added, wherein the conjugate is attached to an anti-biotin antibody or anti-analyte antibody. After complexes are allowed to form the trigger solution is added and detection conducted as above. In another embodiment avidin or streptavidin is deposited on a solid support. A biotin-chemiluminescent compound conjugate is bound to avidin and a biotinylated antibody is also bound. In another embodiment biotin is linked to the solid support and used to capture avidin or streptavidin. A biotinylated antibody is also bound. The chemiluminescent compound can be affixed to the solid support either by binding a biotin-chemiluminescent compound conjugate to the (strept)avidin or by labeling the surface directly with the chemiluminescent compound. Additional analyte-specific binding members known in the art include Fab portion of antibodies, lectin-carbohydrate, protein A-IgG, and hormone-hormone receptor. It is to be understood that indirect binding of chemiluminescent compound to the solid support can be employed in the service of the present disclosure. These and other examples that will occur to one of skill in the art are considered to be within the scope of the present inventive methods.

Solid supports useful in the practice of the present disclosure can be of various materials, porosity, shapes, and sizes. Materials already in use in binding assays including microwell plates of the 96-well, 384-well, or higher number varieties, test tubes, sample cups, plastic spheres, cellulose, paper or plastic test strips, latex particles, polymer particles having diameters of 0.10-50 μm, silica particles having diameters of 0.10-50 μm, magnetic particles, especially those having average diameters of 0.1-10 μm, nanoparticles of various materials, and metal colloids can all provide a useful solid support for attachment of chemiluminescent labels and for immobilizing analyte-specific binding members. Magnetic particles can comprise a magnetic metal, metal oxide or metal sulfide core, which is generally surrounded by an adsorptively or covalently bound layer to shield the magnetic component. The magnetic component can be iron, iron oxide or iron sulfide, wherein iron is Fe²⁺ or Fe³⁺ or both. Usable materials in this class include, e.g., magnetite, maghemite, and pyrite. Other magnetic metal oxides include MnFe₂O₄, Ni Fe₂O₄, and Co Fe₂O₄. The magnetic component can, e.g., be a solid core that is surrounded by a nonmagnetic shell, or can be a core of interspersed magnetic and nonmagnetic material, or can be a layer surrounding a nonmagnetic core, optionally surrounded by another nonmagnetic shell. The nonmagnetic material in such magnetic particles can be silica, synthetic polymers such as polystyrene, Merrifield resin, polyacrylates or styrene-acrylate copolymers, or it can be a natural polymer such as agarose or dextran.

The present disclosure teaches methods of functionalizing such materials for use in the present assay methods. In particular, methods are disclosed for attaching both a chemiluminescent labeling compound and a analyte-specific binding member, such as an antibody, to the same surface, especially to the wells of a microplate or a microparticle. Suitable supports used in assays include synthetic polymer supports, such as polystyrene, polypropylene, substituted polystyrene (e.g., aminated or carboxylated polystyrene), polyacrylamides, polyamides, polyvinylchloride, glass beads, silica particles, functionalized silica particles, metal colloids, agarose, nitrocellulose, nylon, polyvinylidenedifluoride, surface-modified nylon and the like.

Activator Labels

The activator compound forms part of an activator-labeled sbm, which may also be referred to as activator-specific binding member conjugate. The activator-labeled sbm serves a dual function: 1) undergoing a specific binding reaction in proportion to the amount of the analyte in the assay through the specific binding partner portion, either directly or through an intermediary specific binding partner, and 2) activating the chemiluminescent compound through the activator portion. The activator portion of the activator-labeled sbm is a compound that effects the activation of the chemiluminescent compound so that, in the presence of the trigger solution, chemiluminescence is produced. Compounds capable of serving as the activator label include compounds with peroxidase-like activity including transition metal salts and complexes and enzymes, especially transition metal-containing enzymes, most especially peroxidase enzymes. Transition metals useful in activator compounds include those of groups 3-12 of the periodic table, especially iron, copper, cobalt, zinc, manganese, chromium, and vanadium.

The peroxidase enzymes which can undergo the chemiluminescent reaction include e.g., lactoperoxidase, microperoxidase, myeloperoxidase, haloperoxidase, vanadium bromoperoxidase, horseradish peroxidase, fungal peroxidases, lignin peroxidase, peroxidase from Arthromyces ramosus, Mn-dependent peroxidase produced in white rot fungi, and soybean peroxidase. Other peroxidase mimetic compounds are known which are not enzymes but possess peroxidase-like activity including iron complexes, such as heme, and Mn-TPPS₄ (Y.-X. Ci, et al., Mikrochem. J., 52:257-62 (1995)). These catalyze the chemiluminescent oxidation of substrates and are explicitly considered to be within the scope of the meaning of peroxidase as used herein.

In some embodiments, activator-labeled sbm can include conjugates or complexes of a peroxidase and a biological molecule in methods for producing chemiluminescence, the only proviso being that the conjugate display peroxidase or peroxidase-like activity. Biological molecules which can be conjugated to one or more molecules of a peroxidase include DNA, RNA, oligonucleotides, antibodies, antibody fragments, antibody-DNA chimeras, antigens, haptens, proteins, peptides, lectins, avidin, streptavidin and biotin. Complexes including or incorporating a peroxidase, such as liposomes, micelles, vesicles and polymers which are functionalized for attachment to biological molecules, can also be used in the methods of the present disclosure.

Trigger Solutions & Enhancers

The trigger solution provides a reactant necessary for generating the excited state compound necessary for chemiluminescence. The reactant may be one necessary for performing the chemiluminescent reaction by reacting directly with the chemiluminescent label. It may serve instead of or in addition to this function to facilitate the action of the activator compound. This will be the case, for example, when the activator is a peroxidase enzyme. In one embodiment the trigger solution comprises a peroxide compound. The peroxide component is any peroxide or alkyl hydroperoxide capable of reacting with the peroxidase. Exemplary peroxides include hydrogen peroxide, urea peroxide, and perborate salts. The concentration of peroxide used in the trigger solution can be varied within a range of values, typically from about 10⁻⁸ M to about 3 M, more commonly from about 10⁻³ M to about 10⁻¹ M. In another embodiment the trigger solution comprises peroxide and an enhancer compound that promotes the catalytic turnover of an activator having peroxidase activity. A representative embodiment uses a peroxidase conjugate as the activator, an acridan labeled specific binding partner of an analyte wherein the acridan label is provided by reacting the specific binding partner with an acridan labeling compound as described below, and a trigger solution comprising hydrogen peroxide. The peroxide reacts with the peroxidase, presumably to change the oxidation state of the iron in the active site of the enzyme to a different oxidation state. This altered state of the enzyme reacts with an enhancer molecule to promote the catalytic turnover of the enzyme. A reactive species formed from either the enhancer or the enzyme reacts with the acridan label maintained in proximity to the enzyme. The chemiluminescent reaction comprises a further reaction of an intermediate formed from the chemiluminescent compound with peroxide to produce the ultimate reaction product and light.

Incorporation of certain enhancer compounds into the trigger solution promotes the reactivity of the enzyme or reduces background signal or performs both functions. Included among these enhancers are phenolic compounds and aromatic amines known to enhance peroxidase reactions. Mixtures of a phenoxazine or phenothiazine compound with an indophenol or indoaniline compound as disclosed in U.S. Pat. No. 5,171,668 can be used as enhancer in the present invention. Substituted hydroxybenzoxazoles, 2-hydroxy-9-fluorenone, and the compound

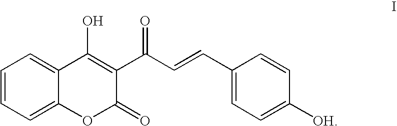

as disclosed in U.S. Pat. No. 5,206,149, can also be used as enhancer in the present invention. Substituted and unsubstituted arylboronic acid compounds and their ester and anhydride derivatives as disclosed in U.S. Pat. No. 5,512,451 are also considered to be within the scope of enhancers useful in the present disclosure. Exemplary phenolic enhancers include but are not limited to: p-phenylphenol, p-iodophenol, p-bromophenol, p-hydroxycinnamic acid, p-imidazolylphenol, acetaminophen, 2,4-dichlorophenol, 2-naphthol and 6-bromo-2-naphthol. Mixtures of more than one enhancer from those classes mentioned above can also be employed.

Additional enhancers that are useful in the practice of the present invention are derivatives include hydroxybenzothiazole compounds and phenoxazine and phenothiazine compounds having the formulas below.

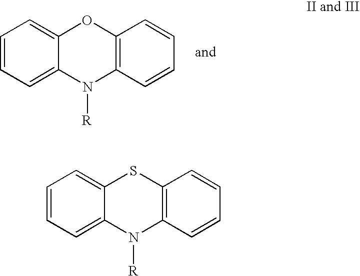

R groups substituted on the nitrogen atom of phenoxazine and phenothiazine enhancers include alkyl of 1-8 carbon atoms, and alkyl of 1-8 carbon atoms substituted with a sulfonate salt or carboxylate salt group. Exemplary enhancers include 3-(N-phenothiazinyl)-propanesulfonic acid salts, 3-(N-phenoxazinyl)propanesulfonic acid salts, 4-(N-phenoxazinyl)butanesulfonic acid salts, 5-(N-phenoxazinyl)-pentanoic acid salts and N-methylphenoxazine and related homologs. The concentration of enhancers used in the trigger solution can be varied within a range of values, typically from about 10⁻⁵ M to about 10⁻¹ M, more commonly from about 10⁻⁴ M to about 10⁻² M.

The detection reaction of the present disclosure is performed with a trigger solution which is typically in an aqueous buffer. Suitable buffers include any of the commonly used buffers capable of maintaining an environment permitting the chemiluminescent reaction to proceed. Typically the trigger solution will have a pH in the range of about 5 to about 10.5. Exemplary buffers include phosphate, borate, acetate, carbonate, tris(hydroxy-methylamino)methane[tris], glycine, tricine, 2-amino-2-methyl-1-propanol, diethanolamine MOPS, HEPES, MES and the like.

The trigger solution can also contain one or more detergents or polymeric surfactants to enhance the luminescence efficiency of the light-producing reaction or improve the signal/noise ratio of the assay. Nonionic surfactants useful in the practice of the present disclosure include by way of example polyoxyethylenated alkylphenols, polyoxyethylenated alcohols, polyoxyethylenated ethers and polyoxyethylenated sorbitol esters. Monomeric cationic surfactants, including quaternary ammonium salt compounds such as CTAB and quaternary phosphonium salt compounds can be used. Polymeric cationic surfactants including those comprising quaternary ammonium and phosphonium salt groups can also be used for this purpose.

In one embodiment the trigger solution is a composition comprising an aqueous buffer, a peroxide at a concentration of about 10⁻⁵ M to about 1M, and an enhancer at a concentration of about 10⁻⁵ M to about 10⁻¹ M. The composition may optionally contain additives including surfactants, metal chelating agents, and preservatives to prevent or minimize microbial contamination.

Specific Binding Pairs

A specific binding pair member or specific binding partner (sbm) is defined herein as a molecule, including biological molecules, having a specific binding affinity for another substance. A specific binding pair member includes DNA, RNA, oligonucleotides, antibodies, antibody fragments, antibody-DNA chimeras, antigens, haptens, proteins, peptides, lectins, avidin, streptavidin and biotin. Each specific binding pair member of a specific binding pair has specific binding affinity for the same substance (e.g. analyte). Each specific binding pair member is non-identical to the other specific binding pair member in a specific binding pair in at least that the specific binding pair members should not compete for the same or overlapping binding site on an analyte. For example, if a specific binding pair is composed of two antibodies, each sbm antibody has a different, non-competing epitope on the analyte.

The specific binding substances include, without limitation, antibodies and antibody fragments, antigens, haptens and their cognate antibodies, biotin and avidin or streptavidin, protein A and IgG, complementary nucleic acids or oligonucleotides, lectins and carbohydrates.

In addition to the aforementioned antigen-antibody, hapten-antibody or antibody-antibody pairs, specific binding pairs also can include complementary oligonucleotides or polynucleotides, avidin-biotin, streptavidin-biotin, hormone-receptor, lectin-carbohydrate, IgG protein A, binding protein-receptor, nucleic acid-nucleic acid binding protein and nucleic acid-anti-nucleic acid antibody. Receptor assays used in screening drug candidates are another area of use for the present methods. Any of these binding pairs can be adapted to use in the present methods by the three-component sandwich technique or the two-component competitive technique described above.

Chemiluminescent Compounds

The compounds used as chemiluminescent labels in the practice of the present disclosure have the general formula CL-L-RG wherein CL denotes a chemiluminescent moiety, L denotes a linking moiety to link the chemiluminescent moiety and a reactive group, and RG denotes a reactive group moiety for coupling to another material. The terms ‘chemiluminescent group’ and ‘chemiluminescent moiety’ are used interchangeably as are the terms ‘linking moiety’ and ‘linking group’. The chemiluminescent moiety CL comprises a compound which undergoes a reaction with an activator resulting in it being converted into an activated compound. Reaction of the activated compound with a trigger solution forms an electronically excited state compound. The excited state may be either a singlet or triplet excited state. The excited state may directly emit light upon relaxation to the ground state or may transfer excitation energy to an emissive energy acceptor, thereby returning to the ground state. The energy acceptor is raised to an excited state in the process and emits light. It is desirable but not necessary, that the chemiluminescent reaction of the CL group, the activator and the trigger solution be rapid, taking place over a very brief time span; in one embodiment reaching peak intensity within a few seconds.

In one embodiment of the disclosure the chemiluminescent compounds are capable of being oxidized to produce chemiluminescence in the presence of the activator and a trigger solution. An exemplary class of compounds which by incorporation of a linker and reactive group could serve as the chemiluminescent label include aromatic cyclic diacylhydrazides such as luminol and structurally related cyclic hydrazides including isoluminol, aminobutylethylisoluminol (ABEI), aminohexylethylisoluminol (AHEI), 7-dimethylaminonaphthalene-1,2-dicarboxylic acid hydrazide, ring-substituted aminophthalhydrazides, anthracene-2,3-dicarboxylic acid hydrazides, phenanthrene-1,2-dicarboxylic acid hydrazides, pyrenedicarboxylic acid hydrazides, 5-hydroxyphthalhydrazide, 6-hydroxyphthalhydrazide, as well as other phthalazinedione analogs disclosed in U.S. Pat. No. 5,420,275 to Masuya et al. and in U.S. Pat. No. 5,324,835 to Yamaguchi.

It is considered that any compound known to produce chemiluminescence by the action of hydrogen peroxide and a peroxidase will function as the chemiluminescent moiety of the chemiluminescent label compound used in the present disclosure. Numerous such compounds of various structural classes, including xanthene dyes such as fluorescein, eosin, rhodamine dyes, or rhodol dyes, aromatic amines and heterocyclic amines are known in the art to produce chemiluminescence under these conditions. Another example is the compound MCLA, 2-methyl-6-(p-methoxyphenyl)-3,7-dihydroimidazo[1,2-a]pyrazin-3-one. Another example is indole acetic acid, another is isobutyraldehyde, the latter typically being accompanied by a fluorescent energy acceptor for increasing the output of visible light. Trihydroxyaromatic compounds pyrogallol, phloroglucinol and purpurogallin, individually or in combination, are other examples of compounds that can serve as chemiluminescent moieties in the chemiluminescent labeling compounds of the disclosure.

In one embodiment a group of chemiluminescent label compounds comprising an acridan ketenedithioacetal (AK) useful in the methods of the disclosure comprises acridan compounds having formula IV

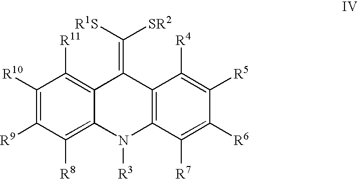

wherein at least one of the groups R¹-R¹¹ is a labeling substituent of the formula -L-RG wherein L is a linking group which can be a bond or another divalent or polyvalent group, RG is a reactive group which enables the chemiluminescent labeling compound to be bound to another compound, R¹, R² and R³ are organic groups containing from 1 to 50 non-hydrogen atoms, and each of R⁴-R¹¹ is hydrogen or a non-interfering substituent. The labeling substituent -L-RG can be present on one of R¹ or R² although it can also be present as a substituent on R³ or one of R⁴-R¹¹.

The groups R¹ and R² in the compound of formula IV can be any organic group containing from 1 to about 50 non hydrogen atoms selected from C, N, O, S, P, Si and halogen atoms which allows light production. By the latter is meant that when a compound of formula I undergoes a reaction of the present disclosure, an excited state product compound is produced and can involve the production of one or more chemiluminescent intermediates. The excited state product can emit the light directly or can transfer the excitation energy to a fluorescent acceptor through energy transfer causing light to be emitted from the fluorescent acceptor. In one embodiment R¹ and R² are selected from substituted or unsubstituted alkyl, substituted or unsubstituted alkenyl, substituted or unsubstituted alkynyl, substituted or unsubstituted aryl, and substituted or unsubstituted aralkyl groups of 1-20 carbon atoms. When R¹ or R² is a substituted group, it can be substituted with 1-3 groups selected from carbonyl groups, carboxyl groups, tri(C₁-C₈ alkyl)silyl groups, a SO₃⁻ group, a OSO₃⁻² group, glycosyl groups, a PO₃⁻ group, a OPO₃⁻² group, halogen atoms, a hydroxyl group, a thiol group, amino groups, C(═O)NHNH₂, quaternary ammonium groups, and quaternary phosphonium groups. In one embodiment, R¹ or R² is substituted with the labeling substituent of the formula -L-RG where L is a linking group and RG is a reactive group.

The group R³ is an organic group containing from 1 to 50 non-hydrogen atoms selected from C, N, O, S, P, Si and halogen in addition to the necessary number of H atoms required to satisfy the valences of the atoms in the group. In one embodiment R³ contains from 1 to 20 non-hydrogen atoms. In another embodiment the organic group is selected from the group consisting of alkyl, substituted alkyl, substituted or unsubstituted alkenyl, substituted or unsubstituted alkynyl, substituted or unsubstituted aryl, and substituted or unsubstituted aralkyl groups of 1-20 carbon atoms. In another embodiment groups for R³ include substituted or unsubstituted C₁-C₄ alkyl groups, phenyl, substituted or unsubstituted benzyl groups, alkoxyalkyl, carboxyalkyl and alkylsulfonic acid groups. When R³ is a substituted group, it can be substituted with 1-3 groups selected from carbonyl groups, carboxyl groups, tri(C₁-C₈ alkyl)silyl groups, a SO₃⁻ group, a OSO₃⁻² group, glycosyl groups, a PO₃⁻ group, a OPO₃⁻² group, halogen atoms, a hydroxyl group, a thiol group, amino groups, C(═O)NHNH₂, quaternary ammonium groups, and quaternary phosphonium groups. The group R³ can be joined to either R⁷ or R⁸ to complete a 5 or 6-membered ring. In one embodiment, R³ is substituted with the labeling substituent of the formula -L-RG.

In the compounds of formula IV, the groups R⁴-R¹¹ each are independently H or a substituent group which permits the excited state product to be produced and generally contain from 1 to 50 atoms selected from C, N, O, S, P, Si and halogens. Representative substituent groups which can be present include, without limitation, alkyl, substituted alkyl, aryl, substituted aryl, aralkyl, alkenyl, alkynyl, alkoxy, aryloxy, halogen, amino, substituted amino, carboxyl, carboalkoxy, carboxamide, cyano, and sulfonate groups. Pairs of adjacent groups, e.g., R⁴-R⁸ or R⁸-R⁶, can be joined together to form a carbocyclic or heterocyclic ring system comprising at least one 5 or 6-membered ring which is fused to the ring to which the two groups are attached. Such fused heterocyclic rings can contain N, O or S atoms and can contain ring substituents other than H such as those mentioned above. One or more of the groups R⁴-R¹¹ can be a labeling substituent of the formula -L-RG. In one embodiment R⁴-R¹¹ are selected from hydrogen, halogen and alkoxy groups such as methoxy, ethoxy, t-butoxy and the like. In another embodiment a group of compounds has one of R⁸, R⁶, R⁹ or R¹⁰ as a halogen and the other of R⁴-R¹¹ are hydrogen atoms.

Substituent groups can be incorporated in various quantities and at selected ring or chain positions in the acridan ring in order to modify the properties of the compound or to provide for convenience of synthesis. Such properties include, e.g., chemiluminescence quantum yield, rate of reaction with the enzyme, maximum light intensity, duration of light emission, wavelength of light emission and solubility in the reaction medium. Specific substituents and their effects are illustrated in the specific examples below, which, however, are not to be considered limiting the scope of the disclosure in any way. For synthetic expediency compounds of formula I desirably have each of R⁴ to R¹¹ as a hydrogen atom.

In another embodiment a group of compounds have formula V wherein each of R⁴ to R¹¹ is hydrogen. The groups R¹, R² and R³ are as defined above.

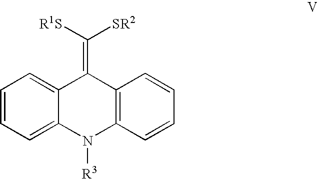

Labeling compounds of formulas IV or V have the groups -L-RG as a substituent on the group R¹ or R². In an embodiment a labeling compound has formula VI.

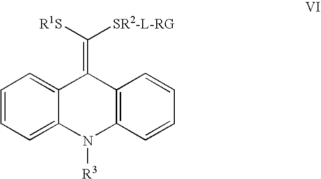

Representative labeling compounds have the structures below. Additional exemplary compounds and their use in attachment to other molecules and solid surfaces are described in the specific examples below. The structures shown below illustrate exemplary compounds of the formula CL-L-RG.

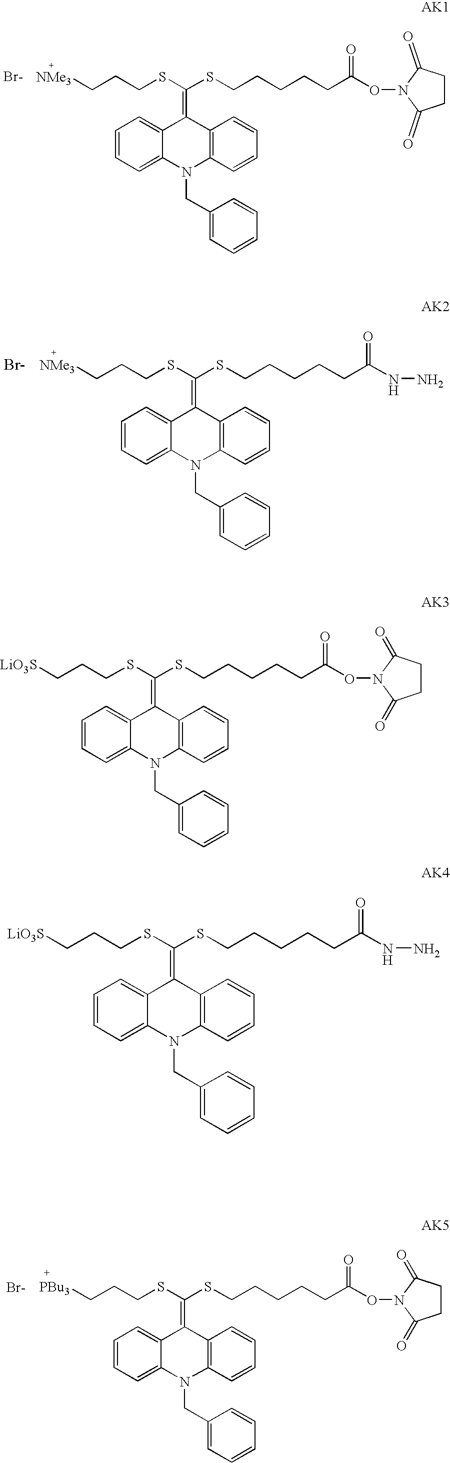

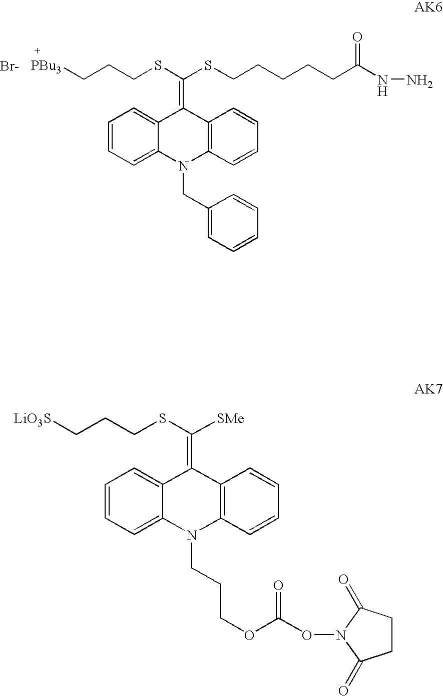

The above specific AK compounds and compounds of general formulas IV, V and VI shown above can be prepared by the skilled organic chemist using generally known methods including methods disclosed in published application US2007/0172878. In an exemplary method an N-substituted and optionally ring-substituted acridan ring compound is reacted with a strong base followed by CS₂ to form an acridan dithiocarboxylate. The dithiocarboxylate is esterified by conventional methods to install one of the substituents designated R¹. The resulting acridan dithioester is again deprotonated with a strong base such as n-BuLi or NaH in an aprotic solvent and S-alkylated with a suitable reagent containing a leaving group and an R² moiety. It will be readily apparent to one of ordinary skill in organic chemistry that the R² moiety may be subject to further manipulation to install suitable reactive groups.

Another class of chemiluminescent moieties includes acridan esters, thioesters and sulfonamides disclosed in U.S. Pat. Nos. 5,491,072; 5,523,212; 5,593,845; and 6,030,803. Chemiluminescent labeling compounds in this class have a chemiluminescent moiety CL of formula VII below wherein Z is O, S or NR¹¹SO₂Ar, wherein R¹¹ is alkyl or aryl, wherein Ar is aryl or alkyl-substituted aryl, wherein R¹ is C₁₋₈ alkyl, halo-substituted C₁₋₈ alkyl, aralkyl, aryl, or aryl substituted with alkyl, alkenyl, alkynyl, aralkyl, aryl, alkoxy, alkoxyalkyl, halogen, carbonyl, carboxyl, carboxamide, cyano, trifluoromethyl, trialkylammonium, nitro, hydroxy, amino and mercapto groups, wherein R² is selected from alkyl, heteroalkyl, aryl, and aralkyl groups, and wherein R³⁻¹⁰ are each hydrogen or 1 or 2 substituents are selected from alkyl, alkoxy, hydroxy, and halogen, and the remaining of R³⁻¹⁰ are hydrogen. In one embodiment each of R³⁻¹⁰ is hydrogen and R¹ is a labeling substituent. In another embodiment one of R³⁻¹⁰ is a labeling substituent and the others of R³⁻¹⁰ are hydrogen.

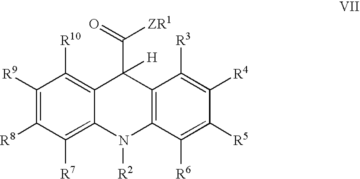

Another class of chemiluminescent moieties includes the heterocyclic compounds disclosed in U.S. Pat. Nos. 5,922,558; 6,696,569; and 6,891,057. In one embodiment the compounds comprise a heterocyclic ring, comprising a nitrogen, oxygen or sulfur-containing five or six-membered ring or multiple ring group to which is bonded an exocyclic double bond, the terminal carbon of which is substituted with two atoms selected from oxygen, and sulfur atoms.

In another embodiment the chemiluminescent labeling compounds comprises a chemiluminescent acridan enol derivative of formula VIII below wherein R¹ is selected from alkyl, alkenyl, alkynyl, aryl, and aralkyl groups of 1-20 carbon atoms any of which can be substituted with 1-3 groups selected from carbonyl groups, carboxyl groups, tri(C₁-C₈ alkyl)silyl groups, a SO₃⁻ group, a OSO₃⁻² group, glycosyl groups, a PO₃″ group, a OPO₃⁻² group, halogen atoms, a hydroxyl group, a thiol group, amino groups, quaternary ammonium groups, or quaternary phosphonium groups, wherein X is selected from C₁-C₈ alkyl, aryl, aralkyl groups, alkyl or aryl carboxyl groups having from 1-20 carbon atoms, tri(C₁-C₈ alkyl)silyl groups, a SO₃⁻ group, glycosyl groups and phosphoryl groups of the formula PO(OR′)(OR″) wherein R′ and R″ are independently selected from C₁-C₈ alkyl, cyanoalkyl, aryl and aralkyl groups, trialkylsilyl groups, alkali metal cations, alkaline earth cations, ammonium and trialkylphosphonium cations, wherein Z is selected from O and S atoms, wherein R⁶ is selected from substituted or unsubstituted C₁-C₈ alkyl, phenyl, benzyl, alkoxyalkyl and carboxyalkyl groups, wherein R⁷⁻¹⁴ are each hydrogen or 1 or 2 substituents are selected from alkyl, alkoxy, hydroxy, and halogen and the remaining of R⁷⁻¹⁴ are hydrogen. In one embodiment each of R⁷⁻¹⁴ is hydrogen and R¹ is a labeling substituent. In another embodiment one of R⁷⁻¹⁴ is a labeling substituent and the others of R⁷⁻¹⁴ are hydrogen.

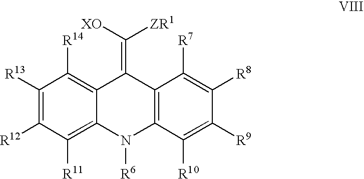

In another embodiment the chemiluminescent labeling compounds comprises a chemiluminescent compound of formula IX below wherein R¹ is selected from alkyl, alkenyl, alkynyl, aryl, and aralkyl groups of 1-20 carbon atoms any of which can be substituted with 1-3 groups selected from carbonyl groups, carboxyl groups, tri(C₁-C₈ alkyl)silyl groups, a SO₃⁻ group, a OSO₃⁻² group, glycosyl groups, a PO₃⁻ group, a OPO₃⁻² group, halogen atoms, a hydroxyl group, a thiol group, amino groups, quaternary ammonium groups, or quaternary phosphonium groups, wherein X is selected from C₁-C₈ alkyl, aryl, aralkyl groups, alkyl or aryl carboxyl groups having from 1-20 carbon atoms, tri(C₁-C₈ alkyl)silyl groups, a SO₃⁻ group, glycosyl groups and phosphoryl groups of the formula PO(OR′)(OR″) wherein R′ and R″ are independently selected from C₁-C₈ alkyl, cyanoalkyl, aryl and aralkyl groups, trialkylsilyl groups, alkali metal cations, alkaline earth cations, ammonium and trialkylphosphonium cations, wherein Z′ and Z² are each selected from O and S atoms and wherein R² and R³ are independently selected from hydrogen and C₁-C₈ alkyl.

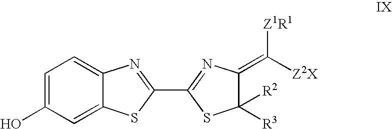

Linking group (L). The linking group in any of the chemiluminescent compounds used in the present disclosure can be a bond, an atom, divalent groups and polyvalent groups, or a straight, or branched chain of atoms some of which can be part of a ring structure. The substituent usually contains from 1 to about 50 non-hydrogen atoms, more usually from 1 to about 30 non-hydrogen atoms. In another embodiment atoms comprising the chain are selected from C, O, N, S, P, Si, B, and Se atoms. In another embodiment atoms comprising the chain are selected from C, O, N, P and S atoms. The number of atoms other than carbon in the chain is normally from 0-10. Halogen atoms can be present as substituents on the chain or ring. Typical functional groups comprising the linking substituent include alkylene, arylene, alkenylene, ether, peroxide, carbonyl as a ketone, ester, carbonate ester, thioester, or amide group, amine, amidine, carbamate, urea, imine, imide, imidate, carbodiimide, hydrazino, diazo, phosphodiester, phosphotriester, phosphonate ester, thioether, disulfide, sulfoxide, sulfone, sulfonate ester, sulfate ester, and thiourea groups. In another embodiment the group is an alkylene chain of 1-20 atoms terminating in a —CH₂—, —O—, —S—, —NH—, —NR—, —SiO—, —C(═O)—, —OC(═O)—, —C(═O)O—, —SC(═O)—, —C(═O)S—, —NRC(═O)—, —NRC(═S)—, or —C(═O)NR— group, wherein R is C₁₋₈ alkyl. In another embodiment the linking group is a poly(alkylene-oxy) chain of 3-30 atoms terminating in a —CH₂—, —O—, —S—, —NH—, —NR—, —SiO—, —C(═O)—, —OC(═O)—, —C(═O)O—, —SC(═O)—, —C(═O)S—, —NRC(═O)—, —NRC(═S)—, or —C(═O)NR— group, wherein R is C₁₋₈ alkyl.

Reactive group. The reactive group RG is an atom or group whose presence facilitates bonding to another molecule by covalent attachment or physical forces. In some embodiments, attachment of a chemiluminescent labeling compound of the present disclosure to another compound or substance will involve loss of one or more atoms from the reactive group for example when the reactive group is a leaving group such as a halogen atom or a tosylate group and the chemiluminescent labeling compound is covalently attached to another compound by a nucleophilic displacement reaction.

In one embodiment RG is an N-hydroxysuccinimide (NHS) ester group. The skilled artisan will readily understand that a substance to be labeled with such a labeling compound comprising an NHS ester group will react with a moiety on the substance, typically an amine group, in the process splitting the ester C—O bond, releasing N-hydroxysuccinimide and forming a new bond between an atom of the substance (N if an amine group) and the carbonyl carbon of the labeling compound.

In another embodiment RG is a hydrazine moiety, —NHNH₂. As is known in the art this group reacts with a carbonyl group in a substance to be labeled to form a hydrazide linkage.

In other embodiments, attachment of a chemiluminescent labeling compound to another compound by covalent bond formation will involve reorganization of bonds within the reactive group as occurs in an addition reaction such as a Michael addition or when the reactive group is an isocyanate or isothiocyanate group. In still other embodiments, attachment will not involve covalent bond formation, but rather physical forces in which case the reactive group remains unaltered. By physical forces is meant attractive forces such as hydrogen bonding, electrostatic or ionic attraction, hydrophobic attraction such as base stacking, and specific affinity interactions such as biotin-streptavidin, antigen-antibody and nucleotide-nucleotide interactions.

Reactive groups for chemical binding of labels to organic and biological molecules include, but are not limited to, the following: a) Amine reactive groups: —N═C═S, —SO₂Cl, —N═C═O, —SO₂CH₂CF₃, N-hydroxysuccinimide ester; b) Thiol reactive groups: —S—S—R; c) Carboxylic acid reactive groups: —NH₂, —OH, —SH, —NHNH₂; d) Hydroxyl reactive groups: —N═C═S, —N═C═O, —SO₂Cl, —SO₂CH₂CF₃; e) Aldehyde/ketone reactive groups: —NH₂, —ONH₂, —NHNH₂; and f) Other reactive groups, e.g., R—N₃,

In one embodiment reactive groups include OH, NH₂, ONH₂, NHNH₂, COOH, SO₂CH₂CF₃, N-hydroxysuccinimide ester, N-hydroxysuccinimide ether and maleimide groups.

Bifunctional coupling reagents can also be used to couple labels to organic and biological molecules with moderately reactive groups (see L. J. Kricka, Ligand-Binder Assays, Marcel Dekker, Inc., New York, 1985, pp. 18-20, Table 2.2 and T. H Ji, “Bifunctional Reagents,” Methods in Enzymology, 91, 580-609 (1983)). There are two types of bifunctional reagents: those that become incorporated into the final structure, and those that do not and serve only to couple the two reactants.

Aqueous Solutions

Aqueous solutions suitable for use in the present disclosure are generally solutions containing greater than 50% water. Aqueous solutions described herein are suitable for uses including reaction mixture, sample dilution, calibrator solutions, chemiluminescent-labeled sbp solutions, activator-labeled sbp solutions, enhancer solutions, and trigger solution, or concentrated solutions of one or more of: chemiluminescent-labeled sbp, activator-labeled sbp, enhancer, trigger, sample, and/or selective signal inhibiting agents. In many embodiments, aqueous solutions are aqueous buffer solutions. Suitable aqueous buffers include any of the commonly used buffers capable of maintaining an environment in aqueous solution maintaining analyte solubility, maintaining reactant solubility, and permitting the chemiluminescent reaction to proceed. Exemplary buffers include phosphate, borate, acetate, carbonate, tris(hydroxy-methylamino)methane (tris), glycine, tricine, 2-amino-2-methyl-1-propanol, diethanolamine MOPS, HEPES, MES and the like. Typically aqueous solutions for use according to the present disclosure will have a pH in the range of about 5 to about 10.5.

Suitable aqueous solutions may include one or more of the following additional components: salts, biological buffers, alcohols, including ethanol, methanol, glycols, and detergents. In some embodiments, aqueous solutions include Tris buffered aqueous solutions, such as Buffer II (Beckman Coulter).

In some embodiments, an aqueous solution emulating human serum is utilized. One such synthetic matrix is 20 mM PBS, 7% BSA, pH 7.5 with 0.1% ProClin 300. Synthetic matrixes can be used for, but not limited to sample dilution, calibrator solutions, chemiluminescent-labeled sbp solutions, activator-labeled sbp solutions, enhancer solutions, and trigger solutions. The term “PBS” refers in the customary sense to phosphate buffered saline, as known in the art. The term “BSA” refers in the customary sense to bovine serum albumin, as known in the art.

Detection

Light emitted by the present method can be detected by any suitable known device or technique such as a luminometer, x-ray film, high speed photographic film, a CCD camera, a scintillation counter, a chemical actinometer or visually. Each detection device or technique has a different spectral sensitivity. The human eye is optimally sensitive to green light, CCD cameras display maximum sensitivity to red light, X-ray films with maximum response to either UV to blue light or green light are available. Choice of the detection device will be governed by the application and considerations of cost, convenience, and whether creation of a permanent record is required. In those embodiments where the time course of light emission is rapid, it is advantageous to perform the triggering reaction to produce the chemiluminescence in the presence of the detection device. As an example the detection reaction may be performed in a test tube or microwell plate housed in a luminometer or placed in front of a CCD camera in a housing adapted to receive test tubes or microwell plates.

Uses

The present assay methods find applicability in many types of specific binding pair assays. Foremost among these are chemiluminescent enzyme linked immunoassays, such as an ELISA. Various assay formats and the protocols for performing the immunochemical steps are well known in the art and include both competitive assays and sandwich assays. Types of substances that can be assayed by immunoassay according to the present disclosure include proteins, peptides, antibodies, haptens, drugs, steroids and other substances that are generally known in the art of immunoassay.

The methods of the present disclosure are also useful for the detection of nucleic acids. In one embodiment a method makes use of enzyme-labeled nucleic acid probes. Exemplary methods include solution hybridization assays, DNA detection in Southern blotting, RNA by Northern blotting, DNA sequencing, DNA fingerprinting, colony hybridizations and plaque lifts, the conduct of which is well known to those of skill in the art.

Assay Materials and Kits

The present disclosure also contemplates providing kits for performing assays in accordance with the methods of the present disclosure. Kits may comprise, in packaged combination, chemiluminescent labels as either the free labeling compounds, chemiluminescent labeled analyte-specific binding members, chemiluminescent derivatized solid supports, such as particles or microplates, or chemiluminescent labeled auxiliary substances such as blocking proteins, along with a trigger solution and instructions for use. Kits may optionally also contain activator conjugates, analyte calibrators and controls, diluents and reaction buffers if chemiluminescent labeling is to be performed by the user.

In another embodiment of the present disclosure there are provided assay materials comprising a solid support having immobilized thereon a chemiluminescent compound. In one embodiment the chemiluminescent compound is selected from any of the group of chemiluminescent compounds described above. In another embodiment the chemiluminescent compound is a substrate for a peroxidase enzyme. The quantity of the chemiluminescent compound immobilized on the solid support can vary over a range of loading densities. As an example, when the solid support is a particulate material, a loading in the range of 100-0.01 μg of chemiluminescent compound per mg of particle can be used. In another example a loading in the range of 5-0.1 μg of chemiluminescent compound per mg of particle can be used. The chemiluminescent compound is generally distributed randomly or uniformly onto the solid support. It may be immobilized on the surface or within accessible pores of the solid support. The chemiluminescent compound can be immobilized onto the solid support by covalent attachment. In this embodiment a chemiluminescent labeling compound having a reactive group is reacted with a functional group present on the solid support in order to form a covalent bond between the chemiluminescent compound and the solid support. In an alternative embodiment the chemiluminescent compound can be immobilized onto the solid support by use of one or more intermediary substances. In one example biotin is covalently attached to the solid support, the covalently attached biotin is bound to streptavidin and a biotin-chemiluminescent compound conjugate is then bound. In another example, streptavidin is adsorbed onto the solid support and a biotin-chemiluminescent compound conjugate is then bound. In another example a chemiluminescent compound conjugated to an auxiliary protein such as albumin is adsorbed or covalently linked onto the solid support. In another example a chemiluminescent compound conjugated to an antibody is adsorbed or covalently linked onto the solid support.

The solid support can be of various materials, porosity, shapes, and sizes such as microwell plates having 96-well, 384-well, or higher numbers of wells, test tubes, sample cups, plastic spheres, cellulose, paper or plastic test strips, latex particles, polymer particles having diameters of 0.10-50 μm, silica particles having diameters of 0.10-50 μm, magnetic particles, especially those having average diameters of 0.1-10 μm, and nanoparticles. In one embodiment the solid support comprises polymeric or silica particles having diameters of 0.10-50 μm, and can be magnetic particles as defined above.

The immobilized chemiluminescent compound of the present disclosure comprises a chemiluminescent label affixed to the solid support wherein the chemiluminescent label is provided by a chemiluminescent labeling compound having the general formula CL-L-RG wherein CL denotes a chemiluminescent moiety, L denotes a linking moiety to link the chemiluminescent moiety to a reactive group, and RG denotes a reactive group moiety for coupling to another material. The chemiluminescent moiety CL comprises a compound which undergoes a reaction with an activator resulting in it being converted into an activated compound. Reaction of the activated compound with a trigger solution forms an electronically excited state compound. The chemiluminescent moiety includes each class of compound described above under the heading “Chemiluminescent Label Compounds” including, without limitation, luminol, and structurally related cyclic hydrazides, acridan esters, thioesters and sulfonamides, and acridan ketenedithioacetal compounds.

In another embodiment of the present disclosure there are provided assay materials comprising a solid support having immobilized thereon a chemiluminescent compound and at least one specific binding substance having specific binding affinity for an analyte or having specific binding affinity for another substance having specific binding affinity for an analyte. In these embodiments the immobilized chemiluminescent compound is as described immediately above for embodiments comprising a solid support having a chemiluminescent compound immobilized thereon. The immobilized specific binding substances directly or indirectly bind an analyte through one or more specific affinity binding reactions. The specific binding substances include, without limitation, antibodies and antibody fragments, antigens, haptens and their cognate antibodies, biotin and avidin or streptavidin, protein A and IgG, complementary nucleic acids or oligonucleotides, lectins and carbohydrates.

Another embodiment of the present disclosure comprises a signaling system formed in an assay comprising a solid support having immobilized thereon 1) a chemiluminescent compound, 2) at least one specific binding substance having specific binding affinity for an analyte or having specific binding affinity for another substance having specific binding affinity for an analyte, 3) an analyte, and 4) an activator conjugate. The meaning of the terms ‘solid support’, ‘chemiluminescent compound’ and ‘specific binding substance’ and embodiments encompassed by these terms are identical to the meanings and embodiments established above for the assay materials considered as compositions of the present disclosure. Analytes that can form an element of the present signaling systems include any of the analytes identified above, the presence, location or amount of which is to be determined in an assay. The activator conjugate comprises an activator compound joined to an analyte-specific binding partner conjugate. The conjugate serves a dual function: 1) binding specifically to the analyte in the assay through the analyte-specific binding member portion, either directly or through an intermediary analyte-specific binding member, and 2) activating the chemiluminescent compound through the activator portion. The activator compound portion of the conjugate is a compound that effects the activation of the chemiluminescent compound so that, in the presence of the trigger solution, chemiluminescence is produced. Compounds capable of serving as the activator include compounds with peroxidase-like activity including transition metal salts and complexes and enzymes, especially transition metal-containing enzymes, especially peroxidase enzymes. Transition metals useful in activator compounds include those of groups 3-12 of the periodic table, especially iron, copper, cobalt, zinc, manganese, and chromium. The peroxidase which can undergo the chemiluminescent reaction include e.g., lactoperoxidase, microperoxidase, myeloperoxidase, haloperoxidase, vanadium bromoperoxidase, horseradish peroxidase, fungal peroxidases, lignin peroxidase, peroxidase from Arthromyces ramosus, Mn-dependent peroxidase produced in white rot fungi, and soybean peroxidase. Other compounds that possess peroxidase-like activity include iron complexes, such as heme, and Mn-TPPS₄.

Systems

The assay methods described in the present disclosure may be automated for rapid performance by employing a system. A system for performing assays of the present disclosure requires the fluid handling capabilities for aliquoting and delivering trigger solution to a reaction vessel containing the other reactants and reading the resulting chemiluminescent signal. In embodiments of such a system, a luminometer is positioned proximal to the reaction vessel at the time and place of trigger solution injection. Additionally, an automated system for performing assays of the present disclosure has fluid handling capabilities for aliquoting and delivering the other reactants and sample to a reaction vessel.

A modified DXI 800 instrument was modified to perform the assay methods of the present disclosure. Further description of the DXI 800 instrument without modification is available in the UniCel DXI User's Guide, ©2007, Beckman Coulter, herein incorporated by reference. For use in performing the methods described herein, a DXI® 800 immunoassay instrument was modified by incorporating a photon-counting luminometer (same model as used in commercially available DXI 800 instrument) positioned for detection near the location of (approximately 19 mm from) the reaction vessel during and immediately after trigger solution injection.

The substrate delivery system within the DXI® 800 immunoassay was used to deliver trigger solution. Some additional components of the DXI® 800 immunoassay instrument not needed for assays according to the methods described herein were removed for convenience, for example magnets and aspiration system used for separation and washing necessary for conventional immunoassay but not used in methods of the present invention. The modified DXI® 800 immunoassay instrument was utilized for convenience in automating reaction vessel handling, pipeting of reagents, detection, and provided temperature control at 37° C. Other commercially available instrumentation may be similarly utilized to perform the assay methods described herein so long as the instrument is able to or may be modified to inject trigger solution into a reaction vessel and start detection of chemiluminescent signal in either a concurrent or nearly concurrent manner. Other example instruments are listed below. The detection of chemiluminescent signal may be of very short duration, several milliseconds, such as one cycle of a photomultiplier tube (PMT) or may be extended for several seconds. All or a portion of the signal collected may be used for subsequent data analysis.

The detection of chemiluminescent signal may be of very short duration, several milliseconds, such as one cycle of a photomultiplier tube (PMT) or may be extended for several seconds. All or a portion of the signal collected may be used for subsequent data analysis. For example, in a typical procedure described below, light intensity is summed for 0.25 sec, centered on the flash of light, in other procedures, light intensity is summed for 5 sec for the first 0.5 sec being a delay before injection.

### EXAMPLES

### Glossary

AHTL: N-acetyl homocysteine lactone

AK: acridan ketenedithioacetal

CKMB: creatine kinase isoenzyme

DMF: dimethyl formamide

EDC: 1-ethyl-3-(3-dimethylaminopropyl) carbodiimide

HRP: horseradish peroxidase

MS-PEG: amine-reactive linear polyethylene glycol polymer with terminal methyl groups

Na2EDTA: sodium salt of ethylene diamine tetraacetic acid.

NHS: N-hydroxysuccinimide

PEG: polyethylene glycol; specifically oligomers or polymers with molecular weight <20,000 g/mol.

PEO: polyethylene oxide; specifically polymers with molecular weight >20,000 g/mol.

PMP: 1-phenyl-3-methyl-5-pyrazolone

PSA: prostate specific antigen

Sulfo-SMCC: Sulfosuccinimidyl-4-(N-maleimidomethyl)cyclohexane-1-carboxylate

TBS: Tris-buffered saline

TnI: Troponin I; cTnI is cardiac Troponin I.

Tris: 2-amino-2-hydroxymethyl-propane-1,3-diol, also known as tris-(hydroxymethyl)aminomethane

Tween®-20: polyoxyethylene(20) sodium monolaurate; commercially available from Sigma-Aldrich, St. Louis (MO).

Materials:

Trigger Solution including Enhancer: An aqueous trigger solution used in many of the examples below, is referred to as Trigger Solution A. Trigger Solution A contains 8 mM p-hydroxycinnamic acid, 1 mM Na₂EDTA, 105 mM Urea Peroxide, 3% ethanol, and 0.2% Tween®-20 in an aqueous buffer solution of 25 mM Tris at pH 8.0. All components are commercially available from various suppliers, such as Sigma, St. Louis, Mo. Buffer II: (TRIS buffered saline, surfactant, <0.1% sodium azide, and 0.1% ProClin® 300 (Rohm and Haas) available commercially from Beckman Coulter, Inc., Brea Calif.).

Instruments:

Modified DxI® 800 Immunoassay Instrument (Beckman Coulter): A modified DXI® 800 instrument was used to perform the assay methods described in several examples below where noted. For use in performing the methods described herein, a DXI® 800 instrument was modified by incorporating a photo-counting luminometer (same model as used in commercially available DXI® 800 instrument) positioned for detection near the location of (approximately 19 mm from) the reaction vessel during and immediately after trigger solution injection. The substrate delivery system within the DXI® 800 immunoassay was used to deliver trigger solution. Some additional components of the DXI® 800 immunoassay instrument not needed for assays according to the methods described herein were removed for convenience, for example magnets and aspiration system used for separation and washing necessary, for conventional immunoassay but not used in methods of the present invention. The modified DXI® 800 immunoassay instrument was utilized for convenience in automating reaction vessel handling, pipeting of reagents, detection, and provided temperature control at 37° C. Other commercially available instrumentation may be similarly utilized to perform the assay methods described herein so long as the instrument is able to or may be modified to inject trigger solution into a reaction vessel and start detection of chemiluminescent signal in either a concurrent or nearly concurrent manner. Other example instruments are listed below. The detection of chemiluminescent signal may be of very short duration, several milliseconds, such as one cycle of a photomultiplier tube (PMT) or may be extended for several seconds. All or a portion of the signal collected may be used for subsequent data analysis.

The detection of chemiluminescent signal may be of very short duration, several milliseconds, such as one cycle of a photomultiplier tube (PMT) or may be extended for several seconds. All or a portion of the signal collected may be used for subsequent data analysis. For example, in a typical procedure described below, light intensity is summed for 0.25 sec, centered on the flash of light, in other procedures, light intensity is summed for 5 sec for the first 0.5 sec being a delay before injection.

Luminoskan Ascent® plate luminometer, (Thermo Fischer Scientific, Inc., Waltham, Mass.) Unmodified. Methods performed at room temperature.

SpectraMax® L microplate luminometer, (Molecular Devices, Sunnyvale, Calif.) Unmodified. Methods performed at room temperature using fast read kinetic mode.

### Example 1

### Selection of SSIA Using Model System

A model system was also developed and employed to screen and select compounds with characteristics to function as selective signal inhibiting agent in assays of the present disclosure. The model system uses a microparticle conjugated to BSA (bovine serum albumin) labeled with a streptavidin and acridan ketenedithioacetal chemiluminescent label (AK1) as the chemiluminescent-labeled sbp, and biotinylated HRP as the activator-labeled specific binding pair. In the model system, varying amounts of Btn-HRP is added to the chemiluminescent-labeled specific binding pair at 0, 1, 10, 100 and 250 ng/mL. Additional unlabeled HRP is added to reach a total HRP of concentration of 500 ng/mL in each reaction mixture. The unlabeled HRP in combination with the activator-labeled sbp was provided to the chemiluminescent-labeled sbp microparticles to emulate sample. A compound for assessment as an SSIA was also added. This reaction mixture of the model system is then triggered by addition of trigger solution in a manner of assays of the present disclosure.

### Preparation of Materials for Model System:

To prepare the chemiluminescent-labeled sbp on microparticles, Bovine Serum Albumin (BSA) was biotinylated with 4× molar excess of biotin-LC-sulfoNHS (Pierce Biotechnology Inc., Rockford, Ill., USA). Unbound reactants were removed via desalting or dialysis. The biotin-BSA was then reacted with a 5× molar excess of acridan ketenedithioacetal AK1 in 20 mM sodium phosphate pH 7.2: DMSO 75:25, v/v) followed by desalting in the same buffer. The dual labeled (biotin and AK1) BSA was then coupled with tosyl activated M-280 microparticles (Invitrogen Corporation, Carlsbad, Calif., USA) in a 0.1 M borate buffer pH 9.5 at a concentration of ca. 20 μg labeled BSA per mg of microparticles for 16-24 h at 40° C. After coupling the microparticles were stripped for 1 h at 40° C. with 0.2 M TRIS base, 2% SDS, pH ˜11. The stripping process was repeated one additional time. Microparticles were then suspended in a 0.1% BSA/TRIS buffered saline (BSA/TBS) buffer and streptavidin (SA) was added at approximately 15 μg SA per mg microparticles. Streptavidin was mixed with the microparticles for 45-50 min at room temperature. The microparticles were then washed three times and suspended in the same BSA/TBS. Studies have shown these base microparticles are capable of binding approximately 5 μg of biotinylated protein per mg of microparticles.

HRP, (Roche Diagnostics, Indianapolis, Ind., USA) was biotinylated with 4× molar excess of biotin-LC-sulfoNHS (Pierce Biotechnology Inc., Rockford, Ill., USA). Unbound reactants were removed via desalting or dialysis.

Each SSIA compound for assessment was dissolved in Buffer II at a concentration at least 10× of final concentration of the reaction mixture (after the addition of the trigger solution)

Paramagnetic particles (PMP): (M280)-(btn-BSA-AK)-(Streptavidin);

Sample Emulator: B-HRP:HRP; 500 ng/mL total with titration of B-HRP:HRP at Total HRP concentration of 500 ng/mL, with Btn-HRP variations: 0, 1, 10, 100 and 250 ng/mL.

SSIA: According to tables below in BUFFER II targeted to give a final concentration of 100 μM.

Trigger solution A is defined above.

### Testing Procedure Using Model System

5 μl of 1 mg/ml of dual-labeled (biotin and AK1) BSA M280 particles were mixed with 45 μl of working concentration SSIA in Buffer II. The assay volume brought to 85 μl by adding 15 μl of Buffer II. 15 μl of sample containing Btn-HRP:HRP at different ratios (The amount of biotinylated-HRP varied from 0, 1, 10, 100 and 250 ng/mL) was added. The reaction mixture was incubated for 30 min at 37° C., then 100 μL of trigger solution was added and the light intensity recorded. Total volume of reaction mixture, including trigger solution was 200 μL with a final concentration of 100 μM of SSIA.

TABLE 2

| | | | | | 5,6iso- | | | |
|---|---|---|---|---|---|---|---|---|
| | | | | Ascorbic | propyliene | (+/−)- | (+)- | |
| | | | Ascorbic | Acid 6- | ascorbic | alpha- | gamma- | |
| | Control | Trolox ® | Acid | palmitate | acid | Tocopherol | Tocopherol | Uric Acid |
| B-HRP0 | 23,603 | 173 | 151 | 81 | 264 | 2,772 | 6,051 | 5,532 |
| B-HRP1 | 45,016 | 1,460 | 995 | 327 | 961 | 6,468 | 10,924 | 9,760 |
| B-HRP10 | 2,149,712 | 37,291 | 32,568 | 40,863 | 29,645 | 1,253,187 | 1,686,079 | 1,025,209 |
| B-HRP100 | 8,926,151 | 7,251,008 | 4,553,473 | 8,187,204 | 4,917,499 | 8,560,469 | 8,698,069 | 8,712,328 |
| B-HRP250 | 9,660,668 | 10,794,247 | 8,915,869 | 10,182,411 | 8,784,595 | 9,628,184 | 10,282,504 | 10,708,733 |
| S/S0 | 1 | 1 | 1 | 1 | 1 | 1 | 1 | 1 |
| S1/S0 | 1.9 | 8.4 | 6.6 | 4 | 3.6 | 2.3 | 1.8 | 1.8 |
| S2/S0 | 91.1 | 215.1 | 216.2 | 502.4 | 112.3 | 452.1 | 278.7 | 185.3 |
| S3/S0 | 378.2 | 41832.7 | 30222.2 | 100662.3 | 18626.9 | 3088.2 | 1437.5 | 1574.9 |
| S4/S0 | 409.3 | 62274.5 | 59176.1 | 125193.6 | 33275 | 3473.4 | 1699.4 | 1935.8 |

| | | | Syringic | |
|---|---|---|---|---|
| | Control | Ferulic acid | Acid | G.W.7.35 |
| B-HRP0 | 27,659 | 5,252 | 14,485 | 67,556 |
| B-HRP1 | 56,887 | 12,079 | 23,707 | 92,403 |
| B-HRP10 | 1,929,315 | 715,313 | 939,372 | 1,600,767 |
| B-HRP100 | 8,598,556 | 8,785,865 | 7,927,096 | 7,938,477 |
| B-HRP250 | 9,255,947 | 10,244,269 | 9,530,979 | 9,509,801 |
| S/S0 | 1 | 1 | 1 | 1 |
| S1/S0 | 2.1 | 2.3 | 1.6 | 1.4 |
| S2/S0 | 69.8 | 136.2 | 64.8 | 23.7 |
| S3/S0 | 310.9 | 1672.9 | 547.2 | 117.5 |
| S4/S0 | 334.6 | 1950.5 | 658 | 140.8 |

TABLE 3

| | | | 4-Amino- | |
|---|---|---|---|---|
| | | | 3-hydroxy- | 4-amino- |
| | | 2-amino- | benzoic | resorcinol |
| | Control | phenol | acid | HCl |
| B-HRP0 | 15,901 | 65 | 972 | 675 |
| B-HRP1 | 46,464 | 356 | 2,808 | 1,163 |
| B-HRP10 | 2,035,193 | 8,632 | 441,455 | 74,764 |
| B-HRP100 | 5,755,341 | 2,092,703 | 5,906,521 | 330,056 |
| B-HRP250 | 6,255,297 | 4,008,689 | 6,403,425 | 259,541 |
| S/S0 | 1 | 1 | 1 | 1 |
| S1/S0 | 2.9 | 5.5 | 2.9 | 1.7 |
| S2/S0 | 128 | 132.8 | 454.2 | 110.8 |
| S3/S0 | 361.9 | 32195.4 | 6076.7 | 489 |
| S4/S0 | 393.4 | 61672.1 | 6587.9 | 384.5 |
| | | | 2-chloro-1,4- | |
| | | 4-chloro- | dihydroxy- | Ascorbic |
| | Control | catechol | benzene | Acid |
| B-HRP0 | 16,161 | 93 | 3,571 | 97 |
| B-HRP1 | 43,300 | 205 | 4,007 | 757 |
| B-HRP10 | 1,769,373 | 1,373 | 188,920 | 16,641 |
| B-HRP100 | 6,027,591 | 456,707 | 610,053 | 3,692,291 |
| B-HRP250 | 6,162,340 | 1,260,937 | 875,831 | 6,036,145 |
| S/S0 | 1 | 1 | 1 | 1 |
| S1/S0 | 2.7 | 2.2 | 1.1 | 7.8 |
| S2/S0 | 109.5 | 14.8 | 52.9 | 171.6 |
| S3/S0 | 373 | 4910.8 | 170.8 | 38064.9 |
| S4/S0 | 381.3 | 13558.5 | 245.3 | 62228.3 |

TABLE 4
INSUFFICIENT EFFECT FOR USE AS SSIA

| | Control | Glutathione | Cysteine | Lipoic Acid |
|---|---|---|---|---|
| B-HRP0 | 30,493 | 26,977 | 35,695 | 35,016 |
| B-HRP1 | 80,841 | 55,719 | 58,203 | 71,751 |
| B-HRP10 | 2,489,892 | 2,480,764 | 2,483,411 | 2,450,949 |
| B-HRP100 | 8,931,915 | 8,733,068 | 9,147,371 | 8,647,037 |
| B-HRP250 | 9,246,768 | 9,965,235 | 10,190,505 | 8,847,921 |
| S/S0 | 1 | 1 | 1 | 1 |
| S1/S0 | 2.7 | 2.1 | 1.6 | 2 |
| S2/S0 | 81.7 | 92 | 69.6 | 70 |
| S3/S0 | 292.9 | 323.7 | 256.3 | 246.9 |
| S4/S0 | 303.2 | 369.4 | 285.5 | 252.7 |

| | | | | | | Nicotinic |
|---|---|---|---|---|---|---|
| | Control | Resveratrol | Melatonin | N-Ac-Cysteine | TEMPOL | Hydrazide |
| B-HRP0 | 30,108 | 64,051 | 54,528 | 43,647 | 22,621 | 42,260 |
| B-HRP1 | 52,680 | 81,452 | 70,741 | 47,636 | 33,873 | 58,356 |
| B-HRP10 | 2,307,964 | 1,073,968 | 2,381,361 | 1,757,607 | 1,963,369 | 2,106,471 |
| B-HRP100 | 8,866,105 | 5,944,792 | 9,471,431 | 8,685,795 | 9,220,799 | 8,205,320 |
| B-HRP250 | 9,055,791 | 6,559,359 | 10,370,061 | 10,219,869 | 10,578,104 | 7,923,092 |
| S/S0 | 1 | 1 | 1 | 1 | 1 | 1 |
| S1/S0 | 1.7 | 1.3 | 1.3 | 1.1 | 1.5 | 1.4 |
| S2/S0 | 76.7 | 16.8 | 43.7 | 40.3 | 86.8 | 49.8 |
| S3/S0 | 294.5 | 92.8 | 173.7 | 199 | 407.6 | 194.2 |
| S4/S0 | 300.8 | 102.4 | 190.2 | 234.2 | 467.6 | 187.5 |

| | | | Acrylamide/bis- | Acrylamide/bis- | |
|---|---|---|---|---|---|
| | | | acrylamide | acrylamide | Nicotinic |
| | Control | Toco-PEG | 19:1 | 37.5:1 | Acid |
| B-HRP0 | 30,608 | 33,836 | 28,760 | 36,028 | 34,369 |
| B-HRP1 | 144,936 | 44,180 | 50,829 | 56,267 | 53,765 |
| B-HRP10 | 2,255,845 | 1,970,753 | 2,286,095 | 2,187,617 | 2,228,317 |
| B-HRP100 | 8,581,227 | 8,352,891 | 8,216,691 | 8,094,544 | 8,772,523 |
| B-HRP250 | 9,183,040 | 9,383,395 | 8,629,933 | 8,463,999 | 9,224,439 |
| S/S0 | 1 | 1 | 1 | 1 | 1 |
| S1/S0 | 1.5 | 1.3 | 1.8 | 1.6 | 1.6 |
| S2/S0 | 73.7 | 58.2 | 79.5 | 60.7 | 64.8 |
| S3/S0 | 280.4 | 246.9 | 285.7 | 224.7 | 255.2 |
| S4/S0 | 300 | 277.3 | 300.1 | 234.9 | 268.4 |

### Conclusions

Compounds demonstrating utility as SSIA include ascorbic acid, 6-palmitate and 5,6-isopropylidene derivatives of ascorbic acid, and TROLOX, a derivative of Tocopherol, 2-aminophenol, 4-amino-3-hydroxybenzoic acid, 4-aminoresorcinol hydrochloride, 4-chlorocatechol, and 2-chloro-1,4-dihydroxybenzene with reductions in background signal indicated by comparing S0 values to the control, and improvements in signal to noise demonstrated by increasing S1/S0 values.

Compounds that have shown no effect in the model system are: glutathione, cysteine, N-acetyl cysteine, lipoic acid (a disulfide), pegylated tocopherol, melatonin (a tryptamine derivative), TEMPOL (a stable nitroxide), nicotinic hydrazide, nicotinic acid, and two acrylamide/bis-acrylamide solutions. A second grouping of compounds, including alpha and gamma-Tocopherol, uric acid, and ferulic acid show a reduction in S0 signal in the range of 75-88%, but do not show an increase in S/S0 until the third calibrator level at 10 ng/mL Btn-HRP.

### Example 2

### Screening SSIA by Homogeneous PSA Immunoassay

This example presents one method used for testing of candidate compounds for functionality as SSIA in assays of the present disclosure. Testing was conducted in a model screening immunoassay of the protein PSA. Mouse anti-PSA tests were run using a 96-well microtiter plate format. A solution containing 30 μL of mouse anti-PSA-AK1 (66 ng), 30 μL of mouse anti-PSA-HRP conjugate (7.8 ng), 36 μL of human female serum, and 244 of PSA calibrator were pipetted into each well. The plate was incubated at 37° C. for 10 minutes. A 5 μL aliquot of the test compound (various concentrations) was added to each well. Chemiluminescence was triggered by the addition of 100 μL of a solution of trigger solution A. The chemiluminescent flash was integrated for 5 seconds after the addition of the trigger solution using a Luminoskan Asent® plate luminometer, (Thermo Fischer Scientific, Inc., Waltham, Mass.).

Each candidate compound was tested at least two levels of PSA: zero and 129 ng PSA/mL (calibrator S5) and/or 2 ng PSA/mL (calibrator S2). For brevity only the results of one representative concentration of each candidate compound are presented. Compounds are considered to be effective at improving assay performance if S5/S0 is improved in relation to a control. It is desirable that the improvement factor be at least 2 (S5/S0≧ about 20-30) and more desirable that improvement factor be at least 5 (S5/S0≧ about 50), yet more desirable that S5/S0 be ≧100 in the present screen. Many compounds were found that exhibited effectiveness as SSIA in this screening test, others were found to be ineffective or have limited effect.

TABLE 5
Test compound, final concentration and S5/S0

| Test Compound | Conc. | | S5/S0 |
|---|---|---|---|
| Control (Serum) | | | 5-10 |
| 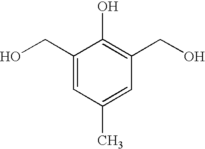 | 0.122 | mM | 23 |
| 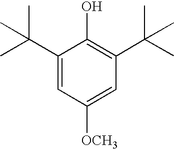 | 0.122 | mM | 19 |
| 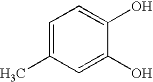 | 0.122 | mM | 142 |
| 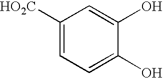 | 0.122 | mM | 178 |
| 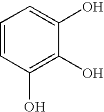 | 0.122 | mM | 69 |
| 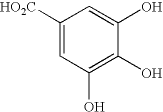 | 0.122 | mM | 135 |
| 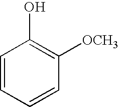 | 0.122 | mM | 13 |
| 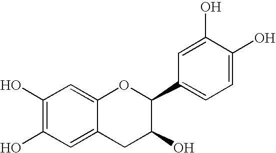 | 0.122 | mM | 323 |
| 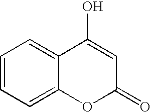 | 0.122 | mM | 17 |
| 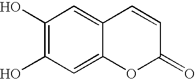 | 0.122 | mM | 105 |
| 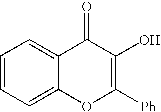 | 0.122 | mM | 13 |
| 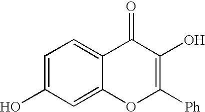 | 0.122 | mM | 14 |
| 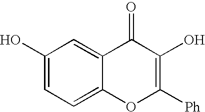 | 0.122 | mM | 9 |
| 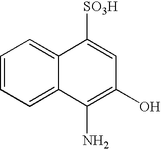 | 0.122 | mM | 65 |
| 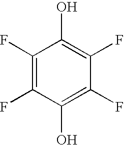 | 0.014 | mM | 50 |
| 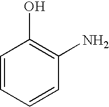 | 0.122 | mM | 649 |
| 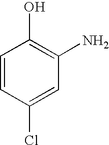 | 0.244 | mM | 205 |
| 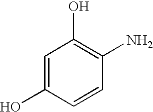 | 0.030 | mM | 161 |
| 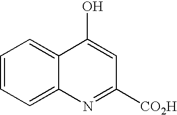 | 0.122 | mM | 6 |
| 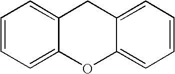 | 0.122 | mM | 4 |
| 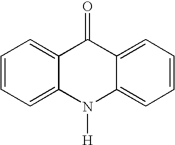 | 0.122 | mM | 14 |
| 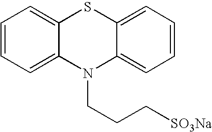 | 0.244 | mM | 50 |
| 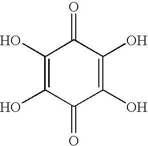 | 0.061 | mM | 51 |
| 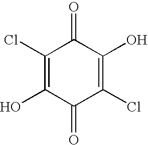 | 0.031 | mM | 7 |
| 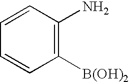 | 0.122 | mM | 108 |
| 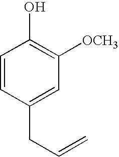 | 0.244 | mM | 30 |
| Glutathione | 122 | uM | 77 |
| L-Cysteine | 122 | uM | 22 |
| NaN3 | 34 | uM | 19 |
| TMB | 61 | uM | 20 |
| 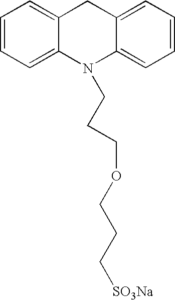 | 0.244 | mM | 6 |
| 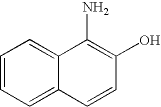 | 0.122 | mM | 63 |
| 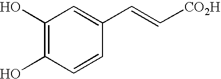 | 0.122 | mM | 116 |
| 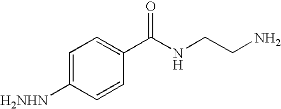 | 1.22 | mM | 138 |
| 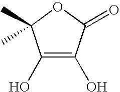 | 0.122 | mM | 467 |
| 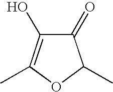 | 1.25 | mM | 237 |
| 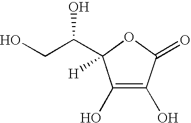 | 122 | μM | 10.3 |
| 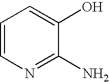 | 0.122 | mM | 32 |
| 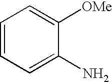 | 0.122 | mM | 7 |
| 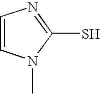 | 36.7 | mM | 102 |
| 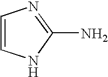 | 24.4 | mM | 14 |
| 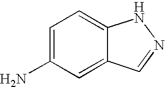 | 12.2 | mM | 10 |
| 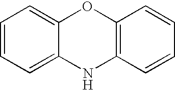 | 0.122 | mM | 605 |
| 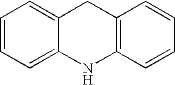 | 0.122 | mM | 120 |
| 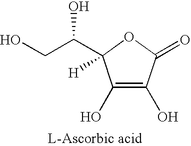 | 0.122 | mM | 423 |
| ascorbate sodium salt (ascorbate anion) | 0.122 | mM | 495 |
| 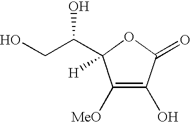 | 122 | uM | 16 |
| 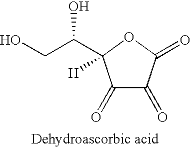 | 0.122 | mM | 26 |
| 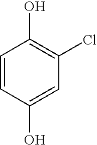 | 0.111 | mM | 229 |
| 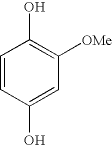 | 0.111 | mM | 161 |
| 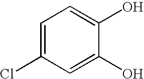 | 0.244 | mM | 409 |
| 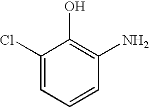 | 0.244 | mM | 300 |
| 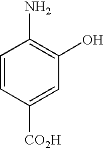 | 0.122 | mM | 153 |
| 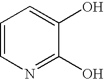 | 0.122 | mM | 41 |
| 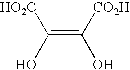 | 0.122 | mM | 30 |
| 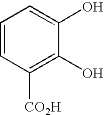 | 0.122 | mM | 22 |
| 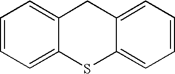 | 0.122 | mM | 9 |
| 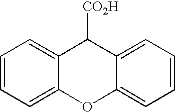 | 0.122 | mM | 7 |
| 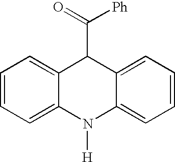 | 0.122 | mM | 9 |
| 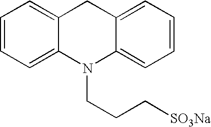 | 0.244 | mM | 15 |
| 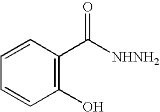 | 0.061 | mM | 23 |
| 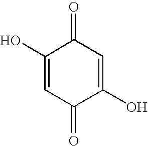 | 0.244 | mM | 14 |
| 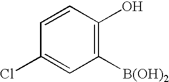 | 0.244 | mM | 22 |
| 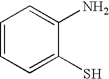 | 0.122 | mM | 234 |
| DTT | 72 | uM | 20 |
| NH2NH2 | 244 | uM | 15 |
| Na2SO3 | 15 | uM | 59 |
| Ethylene glycol | 122 | uM | 14 |
| 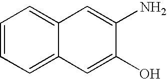 | 0.244 | mM | 67 |
| 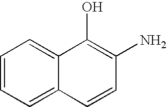 | 0.244 | mM | 109 |
| 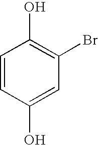 | 0.122 | mM | 570 |
| 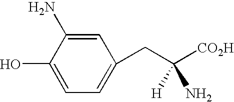 | 0.244 | mM | 448 |
| 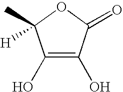 | 0.122 | mM | 423 |
| 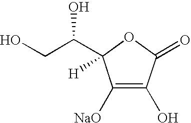 | 122 | μM | 9.2 |
| 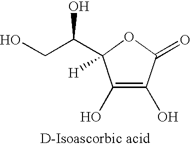 | 122 | μM | 10.3 |

### Example 3

### Preparation of Particles with AK1 and Ab1

This example describes a method for preparing a solid surface (LodeStars™ carboxyl paramagnetic particles, “LodeStar PMP”) with an AK chemiluminescent label and a member of a specific binding pair, Ab1. Ab1 is a monoclonal antibody for an analyte set forth in the subsequent examples (CK-MB, βhCG, myoglobin, cTnI, and PSA). As customary in the art, the term “Ab” optionally followed by a number or letter designator, refers to an antibody with the indicated number or letter designation. Similarly, the term “Ag” refers to antigen in the context of antibody-antigen interaction.

Lodestar PMP (8.33 ml at 30 mg/mL) were suspended in 0.1 M MES/DMSO (75:25) (9.95 ml). EZ-Link Biotin-PEO₄-hydrazide (31.6 μl at 20 mg/mL), EDC (25 mg/mL final concentration), and AK4 having a hydrazide labeling moiety (15.6 μL at 80 mmol/L) were added to the Lodestar PMPs, stirred for 1 minute at 140-160 RPM at room temperature, then overnight (16-24 hours) at 4° C. The particles were then washed and resuspended in BUFFER II. SA21 Streptavidin-Plus (0.49 mL at 10.2 mg/mL) was added to the PMPs to form the AK-Streptavidin Lodestar particles.

Antibodies were biotin labeled using one of the two representative protocols:

1) A 10-fold molar excess of NHS-LC-biotin (Thermo Scientific, Rockford Ill.) was added to anti-cTnI monoclonal antibody, and the mixture was incubated at room temperature for 2 hours. The biotinylated antibody was purified by dialysis in PBS, pH 7.2. The biotin:antibody molar ratio was 4.9, as determined using the commercial biotin quantitation kit (Thermo Scientific), or

2) Biotinylated PSA antibodies were prepared by adding a 6-fold molar excess of NHS-(PEO)4-biotin (Thermo Fisher Scientific, Waltham, Mass.), dissolved in DMSO to 2 mg/mL, to 6 mg of MxPSA antibody (7.6 mg/mL in PBS, pH 7.4). After a 60 min. incubation at ambient temperature, the biotinylated antibody was purified over a Sephadex G-25 column (GE Healthcare, Piscataway, N.J.), equilibrated in PBS, pH 7.4, following the manufacturers instructions.

AK-Streptavidin Lodestar particles (5 mg/mL) were placed in BUFFER II. The needed amount of Ab1 was calculated and added to the AK-Streptavidin Lodestar particles (usually 5 mg/mg, except for βhCG, which was 10 μg/mg). The reaction mixture was vortexed and incubated overnight at 4° C. thereby forming the AK-Abl particle

### Example 4

### Preparation of HRP-Ab2 Conjugate

The HRP-Ab2 conjugates were prepared using known methods in the art. Detailed methods of conjugating HRP to antibodies to produce the HRP-Ab2 conjugates are provided, for example, in the Journal of Immunoassay, Volume 4, Number 3, 1983, p 209-321. Ab2 is a monoclonal antibody for an analyte set forth in the subsequent examples (CK-MB, βhCG, myoglobin, and TnI) that binds to a different antigenic site on the analyte than Ab-1.

Generally, free thiols were attached to the antibody (Ab2) using a product dependent concentration of N-acetyl-DL-homocysteine thiolactone (AHTL). Excess AHTL was removed from the antibody by desalting. Maleimides were attached to the HRP using a molar excess of sulfosuccinimidyl 4-(N-maleimidomethyl)cyclohexane-1-carboxylate (sulfo-SMCC). Excess sulfo-SMCC was removed from the HRP by desalting. The antibody and HRP were combined at a molar ratio of 4 HRP to 1 Ab2 forming a covalent bond between reactant groups. The antibody was metered into the HRP while maintaining the HRP in excess. After incubation for the appropriate amount of time, the reaction was stopped by blocking the unreacted functional groups with β-mercaptoethanol (βME) and N-ethyl maleimide (NEM). The conjugation product (HRP-Ab2 conjugate) was concentrated and separated from any aggregated conjugation products and unreacted antibody or HRP by gel filtration. The conjugation product was pooled based on OD₂₈₀ and OD₄₀₃ activity.

### Example 5

### CK-MB

This example describes a method of detecting CK-MB (Creatine Kinase Myocardial Band) using an AK-Abl particle prepared as set forth in Example 3 and a HRP-Ab2 conjugate prepared as set forth in Example 4 where Ab2 represents an antibody to CK-MB. This method employed ascorbic acid to decrease background signal.

HRP-Ab2 conjugate suspensions were prepared at 1.0 μg/mL and contained either 0 or 1 mM ascorbic acid. Samples consisted of human serum samples with the indicated mounts of CK-MB added or no CK-MB as a control. The test procedure consisted of adding 15 μL of 1.0 μg/mL HRP-Ab2 conjugate and 35 μL of MES buffer containing 1 mg/mL BSA and 1 mg/mL MIgG, pH 5.9 to the reaction vessel. Next, 25 μL of patient serum sample was added, followed by 25 μL of 1.0 mg/mL AK-Abl conjugate suspension thereby obtaining 100 μL of total volume in the reaction vessel. After 15.2 minutes, 100 μL of trigger solution A was added to the reaction vessel and the light intensity was recorded on the modified DxI instrument. Chemiluminescence intensity is expressed in Relative Light Units (RLU).

TABLE 6

| | CK-MB | Without Ascorbate | | | With Ascorbate | | |
|---|---|---|---|---|---|---|---|
| Sample | pg/mL | RLU | Mean | S/S1 | RLU | Mean | S/S1 |
| Buffer | 0 | 172796 | 175047 | | 1616 | 1581 | |
| | | 181300 | | | 1536 | | |
| | | 171044 | | | 1592 | | |
| S1 | 600 | 90872 | 82463 | | 852 | 883 | |
| | | 77216 | | | 848 | | |
| | | 79300 | | | 948 | | |
| S2 | 3200 | 101240 | 103027 | 1.25 | 4080 | 4051 | 4.59 |
| | | 114268 | | | 3928 | | |
| | | 93572 | | | 4144 | | |
| S3 | 9150 | 155396 | 172807 | 2.10 | 12756 | 13295 | 15.06 |
| | | 182036 | | | 13108 | | |
| | | 180988 | | | 14020 | | |
| S4 | 26300 | 544496 | 518571 | 6.29 | 41384 | 40407 | 45.78 |
| | | 508176 | | | 39448 | | |
| | | 503040 | | | 40388 | | |
| S5 | 94600 | 2024460 | 2053723 | 24.90 | 222832 | 232489 | 263.39 |
| | | 2084392 | | | 249468 | | |
| | | 2052316 | | | 225168 | | |
| S6 | 268750 | 3575124 | 3588457 | 43.52 | 1187736 | 1195177 | 1354.05 |
| | | 3566072 | | | 1121256 | | |
| | | 3624176 | | | 1276540 | | |
| S7 | 1500000 | 4113876 | 4131444 | 50.10 | 3266772 | 3324829 | 3766.80 |
| | | 4364516 | | | 3290024 | | |
| | | 3915940 | | | 3417692 | | |

### Example 6

### Beta hCG

This example describes a method of detecting beta-human chorionic gonadotrophin (beta hCG) using an AK-Abl particle prepared as set forth in Example 3 and a HRP-Ab2 conjugate prepared as set forth in Example 4 where Ab2 represents an antibody to beta hCG. This method employed ascorbic acid to decrease background signal.

HRP-Ab2 conjugate suspensions were prepared at 1.0 μg/mL and contained either 0 or 1 mM ascorbic acid. Samples consisted of human serum samples with the indicated mounts of beta hCG added or no beta hCG as a control. The test procedure consisted of adding 20 μL of 1.0 μg/mL HRP-Ab2 conjugate and 30 μL of MES buffer containing 1 mg/mL BSA and 1 mg/mL MIgG, pH 5.9 to the reaction vessel. Next, 25 μL of patient serum sample was added, followed by 25 μL of 10 mg/mL AK-Abl conjugate suspension thereby obtaining 100 μL of total volume in the reaction vessel. After 15.2 minutes, 100 μL of trigger solution A was added to the reaction vessel and the light intensity was recorded on the modified DxI instrument. Chemiluminescence intensity is expressed in Relative Light Units (RLU).

TABLE 7

| | [beta hCG] | | |
|---|---|---|---|
| | (IU/mL) | RLU | S/S0 |
| S0 | 0 | 1,031 | |
| S1 | 4.35 | 7,288 | 7 |
| S2 | 20.81 | 35,320 | 34 |
| S3 | 127.285 | 298,108 | 289 |
| S4 | 413.455 | 1,293,751 | 1,255 |
| S5 | 775.805 | 2,225,779 | 2,159 |

### Example 7

### Myoglobin

This example describes a method of detecting myoglobin using a AK-Abl particle prepared as set forth in Example 3 and a HRP-Ab2 conjugate prepared as set forth in Example 4 where Ab2 represents an antibody to myoglobin. This method employed ascorbic acid to decrease background signal.

HRP-Ab2 conjugate suspensions were prepared at 1.0 μg/mL and contained either 0 or 1 mM ascorbic acid. Samples consisted of human serum samples with the indicated mounts of myoglobin added or no myoglobin as a control. The test procedure consisted of adding 20 μL of 1.0 μg/mL HRP-Ab2 conjugate and 30 μl of MES buffer containing 1 mg/mL BSA and 1 mg/mL MIgG, pH 5.9 to the reaction vessel. Next, 25 μL of patient serum sample was added, followed by 25 μL of 5.0 mg/mL AK-Abl conjugate suspension thereby obtaining 100 μL of total volume in the reaction vessel. After 15.2 minutes, 1004 of trigger solution A was added to the reaction vessel and the light intensity was recorded on the modified DxI instrument. Chemiluminescence intensity is expressed in Relative Light Units (RLU).

TABLE 8

| | [beta hCG] | | |
|---|---|---|---|
| | (ng/mL) | RLU | S/S0 |
| S0 | 11.4 | 1,183 | |
| S1 | 56.3 | 5,557 | 5 |
| S2 | 221 | 31,947 | 27 |
| S3 | 864 | 190,076 | 161 |
| S4 | 2016 | 1,315,465 | 1,048 |
| S5 | 3136 | 4,667,341 | 3,945 |

### Example 8

### cTnI Detection via Heterogeneous Assay

This example describes a method of detecting cTnI (Cardiac Troponin I) using an AK-Abl particle prepared e.g., as set forth in Example 3 and a HRP-Ab2 conjugate prepared e.g., as set forth in Example 4 where Ab2 represents an antibody to cTnI. The effect of ascorbic acid on background signal was investigated.

HRP-Ab2 conjugate suspensions were prepared at 1.0 μg/mL and contained either 0 or 1 mM ascorbic acid. Samples consisted of human serum samples with the indicated mounts of cTnI added or no cTnI as a control. The test procedure consisted of adding 20 μL of 1.0 μg/mL HRP-Ab2 conjugate and 30 μL of MES buffer containing 1 mg/mL BSA and 1 mg/mL MIgG, pH 5.9 to the reaction vessel. Next, 25 μL of patient serum sample was added, followed by 25 μL of 1.0 mg/mL AK-Abl conjugate suspension thereby obtaining 100 μL of total volume in the reaction vessel. After 15.2 minutes, 100 μL of trigger solution A was added to the reaction vessel and the light intensity was recorded on the modified DxI instrument. Chemiluminescence intensity is expressed in Relative Light Units (RLU). Results, provided in the table below, indicate a significant reduction in background signal in the presence of ascorbic acid.

TABLE 9

| | cTnI | | | | |
|---|---|---|---|---|---|
| Std | pg/mL | RLU | S/0 | RLU | S/S0 |
| S0 | 0 | 139008 | | 422 | |
| S1 | 172 | 121418 | 0.9 | 1320 | 3.1 |
| S2 | 366 | 132154 | 1.0 | 2778 | 6.6 |
| S3 | 1368 | 176692 | 1.3 | 10254 | 24.3 |
| S4 | 11136 | 859044 | 6.2 | 108628 | 257.4 |
| S5 | 27922 | 2664610 | 19.2 | 325082 | 770.3 |
| S6 | 106000 | 6130864 | 44.1 | 2380772 | 5641.6 |

### Example 9

### Heterogeneous Assay for GM-CSF

The term “GM-CSF” refers to granulocyte macrophage colon-stimulating factor, a protein necessary for the survival, proliferation and differentiation of hematopoietic progenitor cells, having human gene map locus 5q31.1. A variety of antibodies to GM-CSF are commercially available.

Heterogeneous phase assays directed to GM-CSF were conducted using a LodeStars PMP labeled with AK4 and biotin/streptavidin (AK-PMP-SA) as described in Example 3, an antibody-biotin conjugate and an antibody-HRP conjugate binding to GM-CSF. The antibody-HRP conjugate, (antiGM-CSF-HRP) was purchased from Antigenix.

The antibody-biotin (antiGM-CSF-biotin) conjugate was synthesized by adding a 25-fold molar excess (9.28 μg) of sulfo NHS-biotin (Pierce), dissolved in DMF (1 mg/mL), to 0.1 mg of antibody (Antigenix) in 0.1 mL of 0.1 M sodium borate pH 8.25. After a 60 min incubation at ambient temperature, the reaction was left to incubate overnight at 4° C. The biotinylated antibody was purified over a Sephadex G-25 column (GE Healthcare), equilibrated in PBS, pH 7.4, following the manufacturers instructions.

In order to conduct the heterogeneous assay, 30 μL of an antiGM-CSF-Biotin conjugate solution (0.75 μg/mL, 22.5 ng), 30 μL of calibrator solution having GM-CSF in the range 0-30,000 pg/mL, 30 μL of antiGM-CSF-HRP conjugate (2.25 μg/mL, 67 ng), and 30 μL of AK-streptavidin magnetic particle solution (10 μg of particles) were pipetted into the wells of a white microtiter plate. The plate was incubated for 60 minutes at room temperature. 5 μL of a 2-aminophenol solution (11 mM, 55 nmoles) was added as SSIA. The plate was placed into an injection plate luminometer. 100 μL of trigger solution A was added by the luminometer and the chemiluminescent signal was read for 5 seconds.

The mean intensity of chemiluminescence (RLU), and ratio relative to the absence of GM-CSF in the reaction mixture, (S/S0) as a function of the concentration of GM-CSF in the reaction mixture are provided in the table following.

TABLE 10

| Concentration | Mean | |
|---|---|---|
| (pg/mL) | RLU | S/S0 |
| 30000 | 2180 | 2793.081 |
| 10000 | 580.6 | 743.882 |
| 1000 | 47.89 | 61.358 |
| 100 | 5.2805 | 6.765 |
| 10 | 1.284 | 1.645 |
| 5 | 0.9205 | 1.179 |
| 3 | 0.8865 | 1.135 |
| 1 | 0.8045 | 1.030 |
| 0 | 0.7805 | 1.000 |

### Example 10

### Effect of Trigger Solution pH

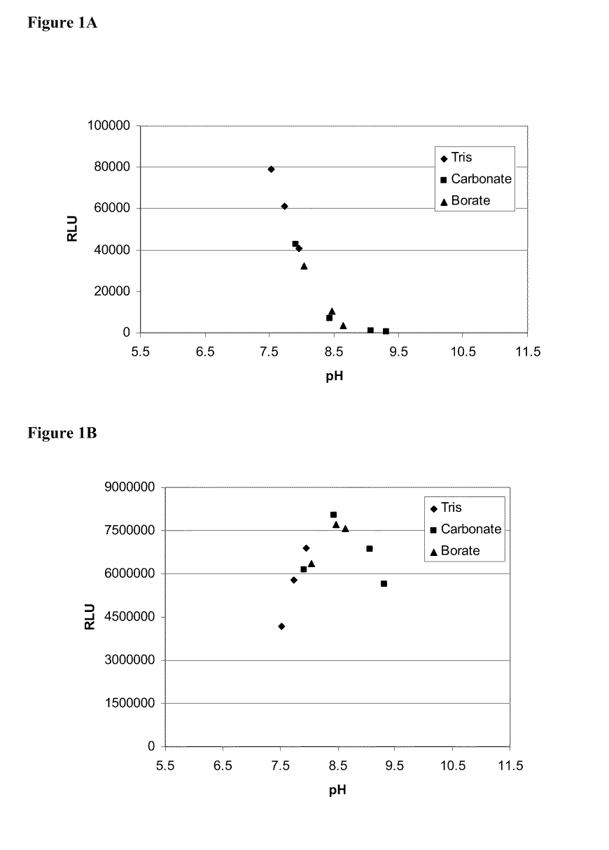

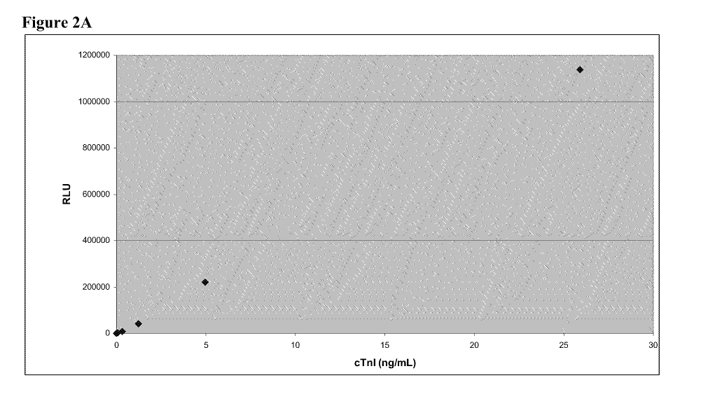

A. The effect of pH on heterogeneous solid-phase assay performance was assessed in a model assay using the biotin-HRP model system of Example 1 on LodeStars particles conjugated directly with AK4 and a biotin hydrazide, as described in Example 3. The particle was then passively overcoated with SA followed by a rinse to remove SA which had not bound biotin, as described above. Buffer salts were selected to afford pH in the range 6-9. The effect on background chemiluminescence of the assay as a function of pH is shown in FIG. 1A. The effect of pH on the specific signal using a ratio of 16:184 btn-HRP:HRP is depicted in FIG. 1B.

B. In order to determine the effect of trigger solution pH on a variety of test assay systems, a series of experiments were conducted varying trigger pH. The following Table 11A provides the average chemiluminescence intensity as a function of PSA concentration in an assay employing PSA on LodeStars particles in the pH range 6.2 to 8.4. Table 11B provides the corresponding results for CK-MB on LodeStar particles in the pH range 5.9 to 8.6. Table 11C provides the corresponding results for TnI on LodeStars particles in the pH range 5.9 to 8.7. In the tables, two pH values are listed for each data set. The first is the pH of the buffer sample added to the reaction mixture. The second is the resulting pH of the final reaction mix.

TABLE 11A
PSA on LodeStar particles with ascorbate

| | PSA | pH 6.0 (6.2) | | pH 7.0 (7.4) | | Control (7.7) | |
|---|---|---|---|---|---|---|---|
| Stnd | pg/ml | Mean RLU | S/S0 | Mean RLU | S/S0 | Mean RLU | S/S0 |
| S0 | 0 | 3,301 | | 7,704 | | 8,192 | |
| S1 | 400 | 34,485 | 10.4 | 87,221 | 11.3 | 91,301 | 11.1 |
| S2 | 1400 | 114,219 | 34.6 | 333,253 | 43.3 | 403,035 | 49.2 |
| S3 | 7000 | 786,859 | 238.3 | 2,640,633 | 342.8 | 2,803,433 | 342.2 |
| S4 | 51000 | 3,198,045 | 968.7 | 9,524,603 | 1236.3 | 10,490,375 | 1280.6 |
| S5 | 101600 | 3,728,345 | 1129.3 | 10,001,948 | 1298.3 | 10,598,780 | 1293.8 |

| | | |
|---|---|---|
| PSA | pH 8.0 (7.9) | pH 9.0 (8.4) |

| Stnd | pg/ml | Mean RLU | S/S0 | Mean RLU | S/S0 |
|---|---|---|---|---|---|
| S0 | 0 | 9,345 | | 5,821 | |
| S1 | 400 | 96,427 | 10.3 | 73,801 | 12.7 |
| S2 | 1400 | 429,327 | 45.9 | 315,528 | 54.2 |
| S3 | 7000 | 3,433,037 | 367.4 | 2,592,673 | 445.4 |
| S4 | 51000 | 11,497,652 | 1230.3 | 11,443,659 | 1965.8 |
| S5 | 101600 | 11,461,215 | 1226.4 | 11,479,280 | 1971.9 |

TABLE 11B
CK-MB on LodeStars with ascorbate

| | CK-MB | pH 6.0 (5.9) | | Control (7.0) | | pH 7.0 (6.8) | |
|---|---|---|---|---|---|---|---|
| Stnd | ng/ml | Mean RLU | S/S0 | Mean RLU | S/S0 | Mean RLU | S/S0 |
| S0 | 600 | 693 | | 1,837 | | 1,788 | |
| S1 | 3200 | 2,849 | 4.1 | 7,380 | 4.0 | 8,409 | 4.7 |
| S2 | 9150 | 6,812 | 9.8 | 22,931 | 12.5 | 22,669 | 12.7 |
| S3 | 26300 | 28,129 | 40.6 | 75,857 | 41.3 | 84,704 | 47.4 |
| S4 | 94600 | 119,144 | 171.8 | 427,483 | 232.7 | 370,193 | 207.0 |
| S5 | 268750 | 754,177 | 1087.8 | 2,055,748 | 1118.9 | 2,084,173 | 1165.6 |

| | | |
|---|---|---|
| CK-MB | pH 8.0 (7.8) | pH 9.0 (8.6) |

| Stnd | ng/ml | Mean RLU | S/S0 | Mean RLU | S/S0 |
|---|---|---|---|---|---|
| S0 | 600 | 3,263 | | 1,512 | |
| S1 | 3200 | 15,725 | 4.8 | 9,508 | 6.3 |
| S2 | 9150 | 42,805 | 13.1 | 28,387 | 18.8 |
| S3 | 26300 | 150,140 | 46.0 | 74,359 | 49.2 |
| S4 | 94600 | 788,451 | 241.7 | 561,103 | 371.1 |
| S5 | 268750 | 4,618,668 | 1415.6 | 3,286,012 | 2173.3 |

TABLE 11C
TnI on LodeStars with ascorbate

| | TnI | pH 6.0 (5.9) | | pH 7.0 (7.1) | | Control (7.5) | |
|---|---|---|---|---|---|---|---|
| Stnd | ng/ml | Ave RLUs | S/S0 | Ave RLUs | S/S0 | Ave RLUs | S/S0 |
| S0 | 0 | 273 | | 519 | | 543 | |
| S1 | 172 | 379 | 1.4 | 1,031 | 2.0 | 1,013 | 1.9 |
| S2 | 366 | 600 | 2.2 | 1,703 | 3.3 | 1,815 | 3.3 |
| S3 | 1368 | 1,452 | 5.3 | 5,695 | 11.0 | 5,981 | 11.0 |
| S4 | 11136 | 11,579 | 42.4 | 57,599 | 111.1 | 53,524 | 98.6 |
| S5 | 27922 | 32,519 | 119.0 | 145,140 | 279.8 | 155,889 | 287.3 |
| S6 | 10600 | 216,379 | 791.6 | 938,075 | 1808.6 | 1,235,103 | 2276.0 |

| | | |
|---|---|---|
| TnI | pH 8.0 (7.9) | pH 9.0 (8.7) |

| Stnd | ng/ml | Ave RLUs | S/S0 | Ave RLUs | S/S0 |
|---|---|---|---|---|---|
| S0 | 0 | 699 | | 413 | |
| S1 | 172 | 1,319 | 1.9 | 716 | 1.7 |
| S2 | 366 | 2,359 | 3.4 | 1,097 | 2.7 |
| S3 | 1368 | 8,048 | 11.5 | 3,999 | 9.7 |
| S4 | 11136 | 72,016 | 103.1 | 36,424 | 88.1 |
| S5 | 27922 | 225,113 | 322.2 | 107,020 | 258.9 |
| S6 | 10600 | 1,574,655 | 2253.8 | 855,956 | 2070.9 |

### Example 11

### Effect of pH on assay signal in PMP model systems

The effect of pH on heterogeneous solid-phase assay performance was further investigated for assays with Dynal M-280 and LodeStars particles in the model system with biotin-HRP as generally described in Example 1. LodeStars particles labeled with AK1-streptavidin-PMP were as described in Example 3. Tosyl activated M-280 particles labeled by covalent coupling with the AK-BSA-biotin as described in Example 1, followed by streptavidin.

### Buffers

With reference to Table 12, buffers were 100 mM in buffer ion, 0.2% in Triton X-100, and 150 mM in NaCl. The “after trigger” pH was determined by combination 1 part buffer, 1 part 25 mM Tris, pH 8, and 2 parts trigger solution A. The temperature for pH reading was 37.4° C.

TABLE 12
Buffers in pH studies

| Sample in cup | After trigger |
|---|---|
| Tris pH 8.0 | 7.53 |
| Tris pH 8.5 | 7.74 |
| Tris pH 9.0 | 7.95 |
| Carbonate pH 9.5 | 7.91 |
| Carbonate pH 10.0 | 8.44 |
| Carbonate pH 10.7 | 9.07 |
| Carbonate pH 11.2 | 9.32 |
| Borate pH 9.4 | 8.04 |
| Borate pH 10.0 | 8.47 |

Sample pH, after trigger pH, relative chemiluminescence and signal-to-noise (S/N) results for this experiment are tabulated in Table 13A and 13B for LodeStars and Dynal M-280 PMPs, respectively,

TABLE 13A
Assay Results for LodeStars PMP

| | pH, | | | | | | | | |
|---|---|---|---|---|---|---|---|---|---|
| | after | RLU | | | | S/N | | | |
| Sample pH | trigger | 0 + 200 | 1 + 199 | 4 + 196 | 16 + 184 | 0 + 200 | 1 + 199 | 4 + 196 | 16 + 184 |
| Tris pH 8.0 | 7.53 | 78882 | 308654 | 1766812 | 4180748 | 1.0 | 3.9 | 22.4 | 53.0 |
| Tris pH 8.5 | 7.74 | 61020 | 349558 | 2258392 | 5780830 | 1.0 | 5.7 | 37.0 | 94.7 |
| Tris pH 9.0 | 7.95 | 40656 | 286812 | 2483580 | 6877444 | 1.0 | 7.1 | 61.1 | 169.2 |
| Carbonate pH | 7.91 | 42920 | 278944 | 2100732 | 6160062 | 1.0 | 6.5 | 48.9 | 143.5 |
| 9.5 | | | | | | | | | |
| Carbonate pH | 8.44 | 6956 | 125000 | 1558166 | 8049738 | 1.0 | 18.0 | 224.0 | 1157.2 |
| 10.0 | | | | | | | | | |
| Carbonate pH | 9.07 | 992 | 43634 | 647858 | 6856012 | 1.0 | 44.0 | 653.1 | 6911.3 |
| 10.7 | | | | | | | | | |
| Carbonate pH | 9.32 | 420 | 26438 | 318140 | 5625568 | 1.0 | 62.9 | 757.5 | 13394.2 |
| 11.2 | | | | | | | | | |
| Borate pH 9.4 | 8.04 | 32490 | 217594 | 1902796 | 6340206 | 1.0 | 6.7 | 58.6 | 195.1 |
| Borate pH | 8.47 | 10274 | 127142 | 1630598 | 7712772 | 1.0 | 12.4 | 158.7 | 750.7 |
| 10.0 | | | | | | | | | |
| Borate pH | 8.64 | 3414 | 87724 | 1179130 | 7586428 | 1.0 | 25.7 | 345.4 | 2222.2 |
| 10.5 | | | | | | | | | |

TABLE 13B
Assay Results for Dynal M-280 PMP

| | pH, | | | | | | | | |
|---|---|---|---|---|---|---|---|---|---|
| | after | RLU | | | | S/N | | | |
| Sample pH | trigger | 0 + 200 | 1 + 199 | 4 + 196 | 16 + 184 | 0 + 200 | 1 + 199 | 4 + 196 | 16 + 184 |
| Tris pH 8.0 | 7.53 | 2712 | 7988 | 143348 | 1628011 | 1.0 | 2.9 | 52.9 | 600.3 |
| Tris pH 8.5 | 7.74 | 1140 | 4339 | 90203 | 1293955 | 1.0 | 3.8 | 79.1 | 1135.0 |
| Tris pH 9.0 | 7.95 | 424 | 2457 | 53768 | 1005841 | 1.0 | 5.8 | 126.8 | 2372.3 |
| Carbonate pH | 7.91 | 544 | 2745 | 63771 | 1152499 | 1.0 | 5.0 | 117.2 | 2118.6 |
| 9.5 | | | | | | | | | |
| Carbonate pH | 8.44 | 101 | 640 | 12492 | 450816 | 1.0 | 6.3 | 123.3 | 4448.8 |
| 10.0 | | | | | | | | | |
| Carbonate pH | 9.07 | 64 | 236 | 1903 | 108417 | 1.0 | 3.7 | 29.7 | 1694.0 |
| 10.7 | | | | | | | | | |
| Carbonate pH | 9.32 | 75 | 157 | 735 | 41728 | 1.0 | 2.1 | 9.8 | 558.9 |
| 11.2 | | | | | | | | | |
| Borate pH 9.4 | 8.04 | 365 | 1776 | 37108 | 770293 | 1.0 | 4.9 | 101.6 | 2108.5 |
| Borate pH 10.0 | 8.47 | 120 | 600 | 10175 | 349089 | 1.0 | 5.0 | 84.8 | 2909.1 |
| Borate pH 10.5 | 8.64 | 89 | 373 | 4833 | 224256 | 1.0 | 4.2 | 54.1 | 2510.3 |

Conclusions

It has been observed that pH greatly affects the chemiluminescence for both LodeStars and Dynal M-280 PMPs, and that the effects are somewhat different between the PMP types.

### Example 12

### Effect of Ascorbic Acid Incubation Time on Chemiluminescence

The effect of the length of time that a sample is exposed to ascorbic acid on the observed reduction of chemiluminescence intensity was investigated in a series of experiments employing the Dynal M-280 PMP particles and biotin-HRP system described in Example 11. Briefly, biotin-labeled PMPs, and various biotin-HRP/HRP solutions were allowed to bind and ascorbic acid solutions added. After a delay period ranging from 80-330 seconds, trigger solution A was injected and the chemiluminescence intensity integrated. The biotin-HRP/HRP solutions contained a total of 200 ng/mL HRP in the proportion 1:200, 8:192, and 32:168 biotin-HRP:HRP. Trials were run using various concentrations of ascorbic acid as the Sample in water at 0, 25, 50 100 and 200 μM.

The results of these investigations demonstrated that ascorbic acid incubation time in the range 80-330 seconds does not appear to cause a significant effect of on the observed chemiluminescence. The result was essentially the same independent of the biotin-HRP/HRP ratio.

### Example 13

### Refinement of Ascorbic Acid Effect on cTnI Assay

The effectiveness of ascorbic acid in improving assay performance in microparticle formats was investigated using a cTnI analyte with various magnetic particles. Magnetic particles evaluated included LodeStars PMP, latex PMP and carboxylate-modified polystyrene latex PMP. “Lot B Magnetic Particle” are 6.2 μm diameter carboxyl PMPs (Bangs Laboratories, Fishers, Ind.). “Lot D Magnetic Particle” are 8.1 μm diameter carboxyl PMPs (Bangs Laboratories). “Lot F Latex Particle” are 3.1 μm diameter carboxyl PMPs (Seradyn Products, Thermo-Fisher, Indianapolis, Ind.). “CML PMP” are 2.9 μM diameter carboxylate modified latex particles (Invitrogen, Carlsbad, Calif.). LodeStars PMP were labeled with AK4 and biotin by EDC coupling and overcoated with streptavidin following the general protocol of Example 3. Lots B, D, and F and CML PMP were labeled with AK-BSA-biotin according to Example 1. The particles were then coated with streptavidin and bound to biotin-labeled anti-cTnI. The experiment protocol was as generally described in Example 8, with an incubation time of 10.2 min. The concentrations of cTnI (i.e., S0-S6) were as provided in Table 9.

### Results

In an initial experiment, the cTnI assay was conducted without ascorbic acid in the reaction mix. As shown in Table 14, LodeStars particles have the highest specific signal; however, background signal overwhelms much of the low calibrator signal.

TABLE 14
cTnI analyte without ascorbate

| | LodeStars ™ | | | Lot B Magnetic Particle | | | Lot D Magnetic Particle | | |
|---|---|---|---|---|---|---|---|---|---|
| [S] | RLU | Mean | % CV | RLU | Mean | % CV | RLU | Mean | % CV |
| s0 | 134464 | 141,229 | 4.7 | 1392 | 1,461 | 6.4 | 700 | 677 | 10.7 |
| | 141600 | | | 1424 | | | 596 | | |
| | 147624 | | | 1568 | | | 736 | | |
| s1 | 166464 | 160,423 | 7.8 | 1752 | 1,741 | 1.3 | 816 | 765 | 5.7 |
| | 146036 | | | 1756 | | | 740 | | |
| | 168768 | | | 1716 | | | 740 | | |
| s2 | 179964 | 184,845 | 7.2 | 1488 | 1,575 | 6.7 | 704 | 732 | 7.1 |
| | 174608 | | | 1544 | | | 700 | | |
| | 199964 | | | 1692 | | | 792 | | |
| s3 | 217724 | 239,016 | 10.2 | 2332 | 2,556 | 15.5 | 920 | 953 | 3.1 |
| | 265504 | | | 3012 | | | 964 | | |
| | 233820 | | | 2324 | | | 976 | | |
| s4 | 983048 | 911,789 | 8.2 | 15908 | 14,475 | 8.6 | 3192 | 3,179 | 2 |
| | 918448 | | | 13740 | | | 3236 | | |
| | 833872 | | | 13776 | | | 3108 | | |
| s5 | 2583452 | 2,451,497 | 5.3 | 63260 | 66,183 | 3.8 | 11412 | 11,271 | 4.6 |
| | 2324052 | | | 67860 | | | 11708 | | |
| | 2446988 | | | 67428 | | | 10692 | | |
| s6 | 6094416 | 6,299,871 | 2.8 | 718172 | 697,660 | 2.7 | 136420 | 132,728 | 2.5 |
| | 6419212 | | | 680764 | | | 131952 | | |
| | 6385984 | | | 694044 | | | 129812 | | |

| | Lot F Latex Particle | | | CML PMP | | |
|---|---|---|---|---|---|---|
| [S] | RLU | Mean | % CV | RLU | Mean | % CV |
| s0 | | 4,690 | 3.9 | 8320 | 7,817 | 5.6 |
| | 4560 | | | 7604 | | |
| | 4820 | | | 7528 | | |
| s1 | 5436 | 5,684 | 4.1 | 8512 | 8,485 | 0.8 |
| | 5716 | | | 8532 | | |
| | 5900 | | | 8412 | | |
| s2 | 6728 | 6,657 | 1.2 | 9448 | 9,525 | 4.4 |
| | 6668 | | | 9980 | | |
| | 6576 | | | 9148 | | |
| s3 | 9568 | 9,851 | 2.6 | 17348 | 16,808 | 3.7 |
| | 9912 | | | 16940 | | |
| | 10072 | | | 16136 | | |
| s4 | 58896 | 61,273 | 3.4 | 176804 | 179,209 | 2.1 |
| | 62820 | | | 183500 | | |
| | 62104 | | | 177324 | | |
| s5 | 256688 | 261,876 | 2.7 | 903084 | 901,085 | 2.6 |
| | 259064 | | | 876460 | | |
| | 269876 | | | 923712 | | |
| s6 | 2213668 | 2,273,623 | 3.3 | 4366112 | 4,425,244 | 3.1 |
| | 2249512 | | | 4583436 | | |
| | 2357688 | | | 4326184 | | |

When the experiment is repeated with ascorbic acid at 150 μM prior to addition of trigger, the results shown in Table 15 are obtained. In this case, LodeStars particles retain much more specific signal than the other particle types, even while the background decreases almost 300%.

TABLE 15
Assay particles with ascorbate at 150 uM.

| | LodeStars | | | Lot B Magnetic Particle | | | Lot D Magnetic Particle | | |
|---|---|---|---|---|---|---|---|---|---|
| [S] | RLU | Mean | % CV | RLU | Mean | % CV | RLU | Mean | % CV |
| S0 | 488 | 533 | 7.7 | 64 | 57 | 14.5 | 56 | 51 | 12.1 |
| | 544 | | | 48 | | | 44 | | |
| | 568 | | | 60 | | | 52 | | |
| S1 | 1360 | 1,415 | 8.2 | 80 | 73 | 11.4 | 52 | 60 | 11.5 |
| | 1548 | | | 76 | | | 64 | | |
| | 1336 | | | 64 | | | 64 | | |
| S2 | 2976 | 2,841 | 4.1 | 72 | 79 | 7.8 | 68 | 59 | 17.2 |
| | 2764 | | | 84 | | | 48 | | |
| | 2784 | | | 80 | | | 60 | | |
| S3 | 9872 | 9,841 | 0.3 | 132 | 129 | 3.6 | 68 | 67 | 15.1 |
| | 9828 | | | 132 | | | 76 | | |
| | 9824 | | | 124 | | | 56 | | |
| S4 | 99432 | 95,989 | 4.8 | 692 | 699 | 3.8 | 176 | 181 | 3.4 |
| | 97780 | | | 728 | | | 180 | | |
| | 90756 | | | 676 | | | 188 | | |
| S5 | 278048 | 276,301 | 4 | 1996 | 1,923 | 3.3 | 412 | 415 | 2 |
| | 286364 | | | 1896 | | | 424 | | |
| | 264492 | | | 1876 | | | 408 | | |
| S6 | 2051508 | 2,121,644 | 3.6 | 16612 | 16,355 | 1.8 | 2508 | 2,445 | 5.5 |
| | 2111244 | | | 16420 | | | 2536 | | |
| | 2202180 | | | 16032 | | | 2292 | | |

| | Lot F Latex Particle | | | CML PMP | | |
|---|---|---|---|---|---|---|
| [S] | RLU | Mean | % CV | RLU | Mean | % CV |
| S0 | 84 | 79 | 21.2 | 68 | 76 | 13.9 |
| | 92 | | | 72 | | |
| | 60 | | | 88 | | |
| S1 | 120 | 135 | 16.4 | 132 | 131 | 4.7 |
| | 124 | | | 136 | | |
| | 160 | | | 124 | | |
| S2 | 240 | 259 | 6.4 | 236 | 237 | 9.3 |
| | 264 | | | 216 | | |
| | 272 | | | 260 | | |
| S3 | 716 | 717 | 0.9 | 780 | 761 | 3.4 |
| | 724 | | | 732 | | |
| | 712 | | | 772 | | |
| S4 | 6528 | 6,509 | 1 | 7792 | 8,012 | 2.4 |
| | 6560 | | | 8108 | | |
| | 6440 | | | 8136 | | |
| S5 | 20760 | 20,592 | 1.5 | 28548 | 28,508 | 2.6 |
| | 20236 | | | 27760 | | |
| | 20780 | | | 29216 | | |
| S6 | 169860 | 176,439 | 5.7 | 341056 | 319,716 | 7.9 |
| | 171456 | | | 326204 | | |
| | 188000 | | | 291888 | | |

### Conclusions

The results provided in this example demonstrate that including ascorbic acid in the assay reaction mixture significantly improves the assay sensitivity.

### Example 14

### Investigation of Effect of Particle Type

In order to further investigate the effect of specific solid phase particles on the assays described herein, a comparison of a variety of particle types was conducted, including silica, polymethylmethacrylate (PMMA). Carboxyl modified PMMA particles (PolyAn GmbH, Berlin) were labeled with AK and biotin as described in Example 3, followed by coating with streptavidin. Silica particles were reacted with 3-aminopropylsiloxane in 1 mM acetic acid to provide an amine reactive group. The amine functional groups were reacted with AK-3 and biotin-LC-sulfoNHS, followed by coating with streptavidin.

Signal generation with Silica and PMMA particles. Assays using the HRP model system, as generally described in Example 1, were conducted on silica particles and PMMA particles, with and without ascorbic acid in the reaction mixture. The assays were run on a modified DxI instrument as described above. The assay conditions consisted of combining 45 μL BUFFER II (with or without ascorbic acid), 25 μL of particle suspension, and 15 μL of sample and incubating for 30 min. Then 100 μL of trigger solution A was added to the reaction vessel and the light intensity was recorded.

The results are provided in Table 15A (silica) and Table 15B (PMMA) with concentration conditions indicated in the tables.

TABLE 16A
Results of silica particles in HRP model system.

| HRP | No Ascorbate | | | 150 μM Ascorbate | | |
|---|---|---|---|---|---|---|
| (ng/ml) | RLU | Mean | S/S0 | RLU | Mean | S/S0 |
| 0 | 117,300 | 128,020 | 1.0 | 256 | 263 | 1.0 |
| | 114,436 | | | 260 | | |
| | 152,324 | | | 272 | | |
| 1 | 189,164 | 173,743 | 1.4 | 7,408 | 6,988 | 26.6 |
| | 163,908 | | | 6,712 | | |
| | 168,156 | | | 6,844 | | |
| 10 | 1,315,928 | 1,302,236 | 10.2 | 310,600 | 309,873 | 1178.2 |
| | 1,293,348 | | | 278,320 | | |
| | 1,297,432 | | | 340,700 | | |
| 100 | 1,273,732 | 1,250,235 | 9.8 | 2,306,036 | 2,426,677 | 9226.9 |
| | 1,274,180 | | | 2,477,760 | | |
| | 1,202,792 | | | 2,496,236 | | |
| 250 | 784,992 | 729,012 | 5.7 | 2,535,712 | 2,533,168 | 9631.8 |
| | 731,048 | | | 2,523,068 | | |
| | 670,996 | | | 2,540,724 | | |

TABLE 16B
Results of PMMA particles in HRP model system.

| HRP | No Ascorbate | | | 200 μM Ascorbate | | |
|---|---|---|---|---|---|---|
| (ng/ml) | RLU | Mean | S/S0 | RLU | Mean | S/S0 |
| 0 | 2,407,880 | 2,359,503 | 1.0 | 20,656 | 20,135 | 1.0 |
| | 2,321,756 | | | 19,436 | | |
| | 2,348,872 | | | 20,312 | | |
| 1 | 3,197,996 | 3,314,221 | 1.4 | 122,512 | 118,472 | 5.9 |
| | 3,476,164 | | | 116,596 | | |
| | 3,268,504 | | | 116,308 | | |
| 10 | 10,720,244 | 10,849,748 | 4.6 | 2,066,824 | 2,061,780 | 102.4 |
| | 11,020,788 | | | 2,171,080 | | |
| | 10,808,212 | | | 1,947,436 | | |
| 100 | 12,019,352 | 11,980,965 | 5.1 | 12,257,256 | 12,093,261 | 600.6 |
| | 12,009,428 | | | 11,862,048 | | |
| | 11,914,116 | | | 12,160,480 | | |
| 250 | 11,804,956 | 11,843,680 | 5.0 | 12,575,032 | 12,565,744 | 624.1 |
| | 11,843,148 | | | 12,552,012 | | |
| | 11,882,936 | | | 12,570,188 | | |

### Example 15

### Investigation of Effect of Particle Type

Signal generation with Silica and PMMA particles. A comparison of Dynal M-280, 3 um CML, 6 um CML, PMMA, silica and LodeStars particles was conducted using the cTnI assay described above. The preparation of each particle, bearing a coating of streptavidin, is described in the foregoing examples. Ascorbic acid, when present, was at 150 μM. The results are provided in Table 17 following. In each particle system tested, the presence of 150 μM ascorbic acid markedly improved S/S0 at the highest calibrator level and, when tested, at the lowest level as well.

TABLE 17
Results of various particles in cTnI assay system.

| | | Dynal | | 3 μm CML | | 6 μm CML | |
|---|---|---|---|---|---|---|---|
| | TnI | No | | No | | No | |
| Cal. | ng/ml | Ascorbate | Ascorbate | Ascorbate | Ascorbate | Ascorbate | Ascorbate |
| S0 | 0 | 1,760 | 58 | 4,690 | 79 | 7,817 | 76 |
| S1 | 0.17 | 2,060 | 108 | 5,684 | 135 | 8,485 | 131 |
| S2 | 0.37 | 2,336 | 144 | 6,657 | 259 | 9,525 | 237 |
| S3 | 1.4 | 4,374 | 446 | 9,851 | 717 | 16,808 | 761 |
| S4 | 11.1 | 63,958 | 4,860 | 61,273 | 6,509 | 179,209 | 8,012 |
| S5 | 27.9 | 333,244 | 16,678 | 261,876 | 20,592 | 901,085 | 28,508 |
| S6 | 106 | 2,070,440 | 171,714 | 2,273,623 | 176,439 | 4,425,244 | 319,716 |

| | | | | | | |
|---|---|---|---|---|---|---|
| S1/S0 | 1.2 | 1.9 | 1.2 | 1.7 | 1.1 | 1.7 |
| S2/S0 | 1.3 | 2.5 | 1.4 | 3.3 | 1.2 | 3.1 |
| S3/S0 | 2.5 | 7.7 | 2.1 | 9.1 | 2.2 | 10.0 |
| S4/S0 | 36.3 | 83.8 | 13.1 | 82.7 | 22.9 | 105.4 |
| S5/S0 | 189.3 | 287.6 | 55.8 | 261.8 | 115.3 | 375.1 |
| S6/S0 | 1176.4 | 2960.6 | 484.8 | 2242.9 | 566.1 | 4206.8 |

| | | PMMA | | Silica | | LodeStars ™ | |
|---|---|---|---|---|---|---|---|
| | TnI | No | | No | | No | |
| Cal. | ng/ml | Ascorbate | Ascorbate | Ascorbate | Ascorbate | Ascorbate | Ascorbate |
| S0 | 0 | 1,074,342 | 173 | 56,458 | 76 | 141,229 | 533 |
| S1 | 0.17 | | 308 | | 100 | 160,423 | 1,415 |
| S2 | 0.37 | | 527 | | 140 | 184,845 | 2,841 |
| S3 | 1.4 | | 1,691 | | 349 | 239,016 | 9,841 |
| S4 | 11.1 | | 14,047 | | 2,717 | 911,789 | 95,989 |
| S5 | 27.9 | | 42,413 | | 7,399 | 2,451,497 | 276,301 |
| S6 | 106 | 8,433,666 | 344,311 | 659,864 | 47,649 | 6,299,871 | 2,121,644 |

| | | | | | | |
|---|---|---|---|---|---|---|
| S1/S0 | | 1.8 | | 1.3 | 1.1 | 2.7 |
| S2/S0 | | 3.0 | | 1.8 | 1.3 | 5.3 |
| S3/S0 | | 9.8 | | 4.6 | 1.7 | 18.5 |
| S4/S0 | | 81.0 | | 35.8 | 6.5 | 180.0 |
| S5/S0 | | 244.7 | | 97.4 | 17.4 | 518.1 |
| S6/S0 | 7.9 | 1986.4 | 11.7 | 627.0 | 44.6 | 3978.1 |

### Example 16

### Investigation of Effect of Ascorbic Acid Concentration

Effect of ascorbic acid on cTnI assay using various particles. The effect of varying the ascorbic acid concentration in the range 150 μM to 9.4 μM on chemiluminescence was investigated for assays employing Dynal M-280, 6 μm CML, and LodeStars particles. The assay for cTnI was as generally described in Example 8, with concentrations as provided in Tables 18A-C. Each particle type tested revealed that all concentrations of ascorbic acid improved analytical sensitivity by increasing signal/background.

TABLE 18A
Results of Dynal M-280 particles in cTnI system.

| | Tnl | Ascorbate Concentration | | | | | |
|---|---|---|---|---|---|---|---|
| Cal. | Ng/ml | No | 150 μM | 75 μM | 38 μM | 19 μM | 9.5 μM |
| S0 | 0 | 1,760 | 58 | 122 | 142 | 296 | 873 |
| S1 | 0.17 | 2,060 | 108 | 190 | 264 | 473 | 1,087 |
| S2 | 0.37 | 2,336 | 144 | 348 | 437 | 736 | 1,539 |
| S3 | 1.4 | 4,374 | 446 | 916 | 1,283 | 1,965 | 3,557 |
| S4 | 11.1 | 63,963 | 4,860 | 9,694 | 14,715 | 25,313 | 46,277 |
| S5 | 27.9 | 333,244 | 16,678 | 38,624 | 69,545 | 146,543 | 297,629 |
| S6 | 106 | 2,070,440 | 171,714 | 586,334 | 1,145,713 | 1869,385 | 2,284,741 |

| | | | | | | |
|---|---|---|---|---|---|---|
| S1/S0 | 1.2 | 1.9 | 1.6 | 2.2 | 1.6 | 1.2 |
| S2/S0 | 1.3 | 2.5 | 2.9 | 3.1 | 2.5 | 1.8 |
| S3/S0 | 2.5 | 7.7 | 7.5 | 8.9 | 6.6 | 4.1 |
| S4/S0 | 36.3 | 83.8 | 79.5 | 103.6 | 85.5 | 53.0 |
| S5/S0 | 189.3 | 287.6 | 316.4 | 489.8 | 495.1 | 340.8 |
| S6/S0 | 1176.4 | 2960.6 | 4806.0 | 8068.4 | 6315.5 | 2616.1 |

TABLE 18B
Results of CML particles in cTnI system.

| | Tnl | Ascorbate Concentration | | | | | |
|---|---|---|---|---|---|---|---|
| Cal. | Ng/ml | No | 150 μM | 75 μM | 38 μM | 19 μM | 9.5 μM |
| S0 | 0 | 7,817 | 76 | 152 | 200 | 377 | 740 |
| S1 | 0.17 | 6,485 | 131 | 232 | 339 | 603 | 1,068 |
| S2 | 0.37 | 9,525 | 237 | 380 | 569 | 956 | 1,524 |
| S3 | 1.4 | 16,803 | 761 | 1,267 | 1,785 | 2,824 | 4,188 |
| S4 | 11.1 | 179,209 | 8,012 | 14,117 | 22,248 | 39,588 | 63,926 |
| S5 | 27.9 | 901,085 | 28,508 | 53,496 | 107,569 | 260,602 | 430,072 |
| S6 | 106 | 4,425,244 | 319,716 | 1,115,605 | 2,393,047 | 3,579,441 | 3,799,669 |

| | | | | | | |
|---|---|---|---|---|---|---|
| S1/S0 | 1.1 | 1.7 | 1.5 | 1.7 | 1.6 | 1.4 |
| S2/S0 | 1.2 | 3.1 | 2.5 | 2.8 | 2.5 | 2.1 |
| S3/S0 | 2.3 | 12.0 | 6.3 | 8.9 | 7.5 | 5.7 |
| S4/S0 | 22.9 | 125.4 | 92.9 | 111.2 | 104.9 | 86.4 |
| S5/S0 | 115.3 | 375.1 | 351.8 | 537.8 | 690.6 | 581.2 |
| S6/S0 | 566.1 | 4226.8 | 7339.5 | 11965.2 | 9486.2 | 5134.7 |

TABLE 18C
Results of LodeStars ™ particles in cTnI system.

| | Tnl | Ascorbate Concentration | | | |
|---|---|---|---|---|---|
| Cal. | ng/ml | No | 75 μM | 150 μM | 250 μM |
| S0 | 0 | 139026 | 982 | 526 | 358 |
| S1 | 0.17 | 121416 | 2324 | 1546 | 1296 |
| S2 | 0.37 | 132154 | 4986 | 3434 | 2738 |
| S3 | 1.4 | 176692 | 17432 | 12666 | 9454 |
| S4 | 11.1 | 859044 | 183518 | 126430 | 99090 |
| S5 | 27.9 | 2664610 | 595114 | 362253 | 288992 |
| S6 | 106 | 6130864 | 4212104 | 2786328 | 2078202 |

| | | | | |
|---|---|---|---|---|
| S1/S0 | 0.9 | 2.4 | 3.0 | 3.6 |
| S2/S0 | 1.2 | 5.1 | 6.8 | 7.6 |
| S3/S0 | 1.3 | 17.8 | 25.2 | 26.4 |
| S4/S0 | 6.2 | 187.3 | 248.9 | 276.8 |
| S5/S0 | 19.2 | 627.3 | 752.1 | 607.2 |
| S6/S0 | 44.1 | 4298.1 | 5484.9 | 5605.0 |

### Example 17

### Comparison of Methods for cTnI Analysis: Assay Linearity

Modifications of the assays procedures described herein, including but not limited to the inclusion of additional reagents for reducing background signal, decreasing the time required for assay, eliminating unwanted chemical interactions, and the like, are available to the skilled artisan. Accordingly, in order to further characterize methods for analyte detection as described herein, a series of experiments were conducted wherein the assay components were as described below.

The PMP were LodeStars at 1 mg/mL in 100 mM Tris, 0.15M NaCl, 0.1 mM EDTA, 0.2% Tween 20, 1% BSA, 0.1% Proclin, pH 8.0, conjugated with AK1 and antibody (Abl) to cTnI, prepared by the general procedure in Example 3. HRP-Ab2 conjugate, obtained with Lightning-Link™ methodology (Novus Biologicals, Littleton, Colo.) according to the manufacturer's protocol, was used at 1 μg/mL in combination with 50 μg/mL PolyMak-33 (Roche), 1 mg/mL MIgG (Murine IgG), and 0.5 M NaCl. Standard TnI solutions (SCIPAC) and normal clinical human samples were provided. TnI values of calibrators were determined by AccuTnI assay (Beckman coulter).

The cTnI assay protocol consisted of pipetting 254 of the 1 mg/mL AK-Abl particle suspension, 45 μL of 333 μM ascorbic acid, 15 μL of 1 μg/mL HRP-Ab2, and 15 μL sample. The mixture was incubated for five minutes at 37° C. and then trigger by injection of 100 μL of trigger solution A. The resultant flash of light is measured over 250 milliseconds starting immediately upon trigger addition.

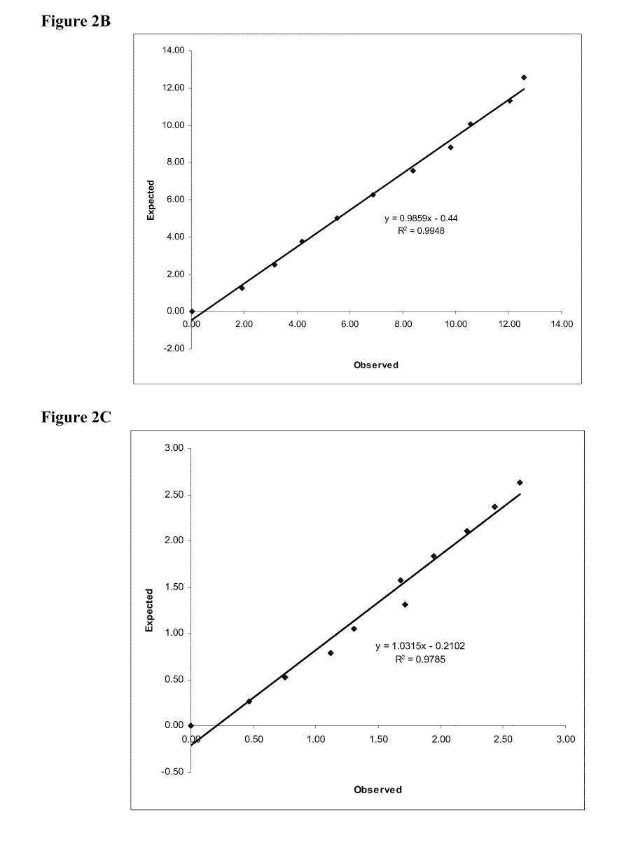

In a representative experiment with results depicted in FIG. 2A, a series of cTnI calibration standards (concentration range: zero to 25.92 ng/mL) were analyzed by the procedure described above. A linear result (R²=0.9999) is observed in this concentration range under the experimental conditions.

The effect of sample dilution on the linearity of response was investigated by diluting a positive cTnI sample (2×) and then systematically diluting the samples in the series 0:10, 1:9, 2:8, 3:7, 4:6, 5:5, 6:4, 7:3, 8:2, 9:1 and 10:0. At the 10:0 dilution (i.e., largest cTnI concentration), the absolute RLU value was 418,256 which corresponds to a concentration of about 9.6 ng/mL. As shown in FIG. 2B, good linearity (i.e., R²=0.9948) is found between observed and expected RLU values under these dilution conditions.

The dilution test protocol was further investigated by the use of an 8× dilution of a positive cTnI sample with the systematic dilution scheme described for FIG. 11B. Under these conditions, the 8× diluted sample provided an RLU value of about 76,832, which corresponds to a concentration of cTnI of about 1.8 ng/mL. As shown in FIG. 2C, even under such dilution conditions, reasonable linearity (R²=0.9785) is observed.

Analytical sensitivity of the assay was measured by generating 20 replicates of the zero analyte calibrator and subsequent calculation of the 2× standard deviation. The 2× standard deviation was projected as a swath on a calibration curve collected with known concentrations of cTnI, providing an estimate of the sensitivity of 0.005 ng/mL cTnI for the procedure.

### Example 18

### Comparison of cTnI Analysis Methods with Access AccuTnI

The results of a cTnI assay conducted by the methods of the present invention were compared with the results of a reference method, the Access AccuTnI system (Beckman Coulter). The present method was performed as described in the previous example with the exception that the ascorbic acid reagent was added as 45 μL of 500 μM ascorbic acid in the reaction mix.

Clinical samples were obtained as follows: no analyte (cTnI) present (25 samples), positive lithium heparin plasma patient samples, positive serum, and matched plasma and serum samples from the same patients (N=15). Standard cTnI solutions were employed, providing cTnI dosing in the range 0 to 17.48 ng/mL.

Analyses of 95 clinical samples, including 54 plasma samples and 41 serum samples, were conducted using the procedure described above (3 replicates), and the Access AccuTnI procedure (2 replicates). Access AccuTnI system results were obtained following manufacturer's instructions. Analyte concentrations for the current procedure were made by comparison with standard calibrator concentrations of cTnI.

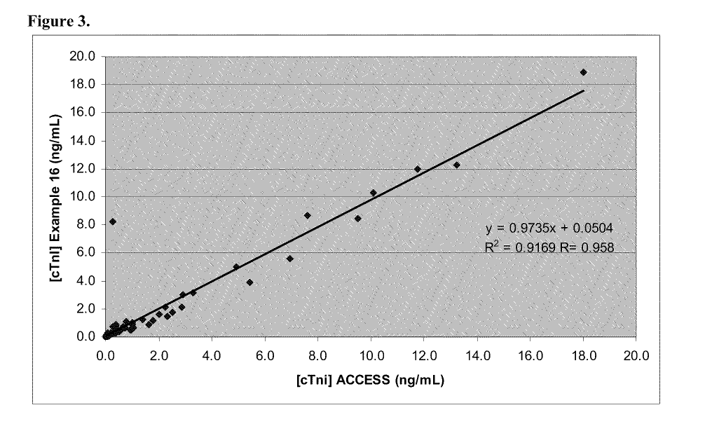

A scatter diagram of the paired results for the current procedure and the Access AccuTn procedure is depicted in FIG. 3. In the figure, the ordinate is the concentration of cTnI observed with the current procedure, and the abscissa is the corresponding concentration of cTnI determined with the Access AccuTnI procedure. A Deming regression analysis, as known in the art, of the data provided in FIG. 12 yielded R²=0.9169 and R=0.958 (N=95).

### Example 19

### Heterogeneous Assay for a DNA Analyte

Heterogeneous phase assays employing magnetic particles and directed to the 2868 base pair pUC18 plasmid DNA were conducted using a paramagnetic particle labeled with AK and Streptavidin (AK-PMP-SA), two biotinylated capture oligonucleotides, a set of fluorescein-labeled reporter oligonucleotides, and an antifluorescein-HRP conjugate. The AK-streptavidin paramagnetic particle conjugate was made as generally described in Examples 1 and 2. The biotin and fluorescein-labeled oligonucleotides were prepared by custom synthesis and designed to be complementary to the template. The antibody-HRP conjugate, was available commercially (Roche). Human gDNA (Roche) was used as a negative control. Annealing buffer contained 10 mM TRIS.Cl pH 8.3, 50 mM KCl, and 1.5 mM MgCl₂. Hybridization buffer contained 6×SSC pH 7 (Sodium chloride/sodium citrate-pH adjusted with NaOH), 0.1% SDS, 24% formamide, 0.37% acetic acid, and 1 μg/mL biotin.

Procedure

1. Binding biotin-labeled oligos to particles. The two oligos (10 μL each of 100 ng/μL solutions) and particles (1 μL of a 5 μg/μL suspension of LodeStars) in 150 μL of 1×PBS buffer, pH 7.4 were vortex mixed and placed in a shaker incubator at 37° C. for 30 min. The particles were pulled to the side of the tube on a magnet and the supernatant discarded. The particles were washed twice with 1×PBS containing 0.05% Tween-20 The particles were resuspended in 140 μL of annealing buffer and aliquotted at 20 μL/tube into six 1.5 mL microfuge tubes labeled 1 through 6.

2. Oligonucleotide-template hybridization and capture. The following annealing reactions were set up in 250 μL tubes. The tubes were heated at 95° C. for 5 min and held at 50° C. After 5 min. at 50° C., 200 μL of hybridization buffer was added to each tube and mixed. The annealing reactions were transferred to the correspondingly numbered 1.5 mL tubes containing 20 μL of particles bound to the biotin-labeled oligos. The mixtures were hybridized in a shaker incubator at 37° C. for 1 hour. The tubes were placed on a magnet for 1 min, and the hybridization buffer was removed.

The particles were washed three times to remove unbound nucleic acid by resuspension in 1×PBS with 0.05% Tween-20, with magnetic separation.

TABLE 19A

| | | Tubes | | | | | |
|---|---|---|---|---|---|---|---|
| | | | | | | | 6 |
| | | 1 | 2 | 3 | 4 | 5 | Neg. |
| pUC 18 | | 20 pg | 2 pg | 200 fg | 20 fg | 2 fg | control |
| Nuclease free | (μL) | 9 | 9 | 9 | 9 | 9 | 11 |
| H₂O | | | | | | | |
| 10X annealing | (μL) | 2 | 2 | 2 | 2 | 2 | 2 |
| buffer | | | | | | | |
| Equal mix of | (μL) | 5 | 5 | 5 | 5 | 5 | 5 |
| 5 FAM oligos | | | | | | | |
| (10 ng/μL) | | | | | | | |
| Human genomic | (μL) | 2 | 2 | 2 | 2 | 2 | 2 |
| DNA 200 ng/μL | | | | | | | |
| pUC18 | (μL) | 2 | 2 | 2 | 2 | 2 | 0 |
| Total | (μL) | 20 | 20 | 20 | 20 | 20 | 20 |

3. Binding anti-fluorescein-HRP antibody to hybridized fluorescein oligos The washed particles from step 2 were resuspended in 1:150,000 dilution of anti-fluorescein-HRP antibody, and incubated at room temperature for 30 min with gentle shaking. Unbound antibody was removed by magnetic separation. The particles were washed three times by resuspension in 1×PBS with 0.05% Tween-20, holding on a magnet for 1 min., and removing the wash buffer.

4. Chemiluminescent SPARCL Detection. The washed particles from step 3 were resuspended in 100 μL of 1×PBS. The particles were split equally (˜48 μL each) into two wells of a white microtiter plate (Nunc). Chemiluminescence was measured by placing the plate in a Luminoskan luminometer (Labsystems), injecting 100 μL of trigger solution (25 mM TRIS pH8.0, 0.1% Tween-20, 1 mM EDTA, 8 mM p-hydroxycinnamic acid, 100 mM urea peroxide) and reading for 5 sec immediately on injection

TABLE 19B

| | Beads | | | | | |
|---|---|---|---|---|---|---|
| | in unblocked | | | | | |
| pUC18 | strips | | Average | S/N | SD | % CV |
| 0 | 0.28 | 0.41 | 0.34 | | 0.09 | 26.96 |
| 1 fg | 0.77 | 0.68 | 0.72 | 2.12 | 0.06 | 8.20 |
| 10 fg | 1.02 | 0.95 | 0.99 | 2.89 | 0.05 | 5.03 |
| 100 fg | 1.28 | 1.18 | 1.23 | 3.60 | 0.07 | 5.99 |
| 1 pg | 3.84 | 4.36 | 4.10 | 12.03 | 0.37 | 9.03 |
| 10 pg | 20.34 | 23.66 | 22.00 | 64.52 | 2.35 | 10.67 |

### Example 20

### Preparation of Silica Particles Labeled with an AK Chemiluminescent Label and Anti-PSA Antibody

Silica particles (5.0 g) derivatized with 3-aminopropylsiloxane (Silicycle Quebec City, Canada) were reacted with AK labeling compound AK3 (2.5 mg) and 1 mL of triethylamine in 50 mL of DMF with stirring under Ar over night. The mixture was filtered and the particles washed with DMF and then with 1:1 CH₂Cl₂/MeOH before air drying. The starting particles contained 1.77 mmol/g of NH₂×5 g=8.85 mmol of NH₂. AK label compound used was 2.5 mg/659 mg/mmol=3.8 μmol. Label incorporation via formation of the amide bond was, therefore, less than 0.05% of the available NH₂ groups.

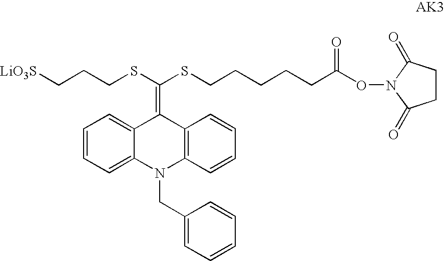

A 25 mg portion of the AK-labeled particle was added to 1.0 mL of DMF containing 2% triethylamine in a microfuge tube, the tube shaken for 10 minutes and the solvent decanted. Particles were washed with DMF and suspended in a solution of 50 mg of the bifunctional linker DSS in 1.2 mL of DMF. After a 30 min incubation on a shaker, the solution was decanted and the particles washed with DMF. The activated particles were bound to mouse anti-PSA (MxPSA, Beckman) by reacting with a solution of 25 μL of 9.0 mg/mL antibody stock diluted in 1.0 mL of pH 8.25 borate buffer at 4° C. for 20 hours.

### Example 21

### Heterogeneous PSA Immunoassay with Chemiluminescent Detection and Et₂NOH as SSIA

Materials

Labeled particles of Example 19 (3.3 mg/mL solution in 1×PBS)

Assay buffer: 0.2% BSA, 0.2% sucrose, 0.2% Tween-20 in 1×PBS

Et₂NOH 9.73 mM solution in 1×PBS

MxPSA-HRP conjugate (0.0152 μg/mL in Assay buffer)

PSA calibrators: S0, S1, S5

Trigger Solution A (Example 1)

Tubes previously blocked with 0.2% BSA, 0.2% sucrose, in 1×PBS were charged with 30 μL of labeled particles, 30 μL of MxPSA-HRP conjugate, 36 μL of Assay buffer, 24 μL of PSA calibrator, and 20 μL of Et₂NOH solution. Single tubes were placed in a luminometer with computer-controlled injection and data collection. Trigger solution A (100 μL) was injected and light intensity summed for 5 sec, the first 0.5 sec being a delay before injection. Results are average of duplicate measurements.

TABLE 20

| | |
|---|---|
| w/o Et₂NOH | w/o Et₂NOH |

| | RLU | S/S0 | RLU | S/S0 |
|---|---|---|---|---|
| S0 | 15368 | — | 6694 | — |
| S1 | 680002 | 44 | 446120 | 66.6 |
| S5 | 54298678 | 3533 | 43144054 | 6445 |

All publications and patent applications in this specification are indicative of the level of ordinary skill in the art to which this invention pertains and are incorporated herein by reference in their entireties and for all purposes.

The various embodiments described above are provided by way of illustration only and should not be construed to limit the invention. Those skilled in the art will readily recognize various modifications and changes that may be made to the present invention without following the example embodiments and applications illustrated and described herein, and without departing from the true spirit and scope of the present invention without following the example embodiments and applications illustrated and described herein, and without departing from the true spirit and scope of the present invention, which is set forth in the following claims.

The claimed invention is:

1. An assay method for an analyte in a sample, the assay method comprising:
forming a reaction mixture in an aqueous solution, in any order or concurrently, by adding:
a chemiluminescent-labeled immobilized specific binding member including a solid support, including a first analyte-specific binding member conjugated to the solid support, and a chemiluminescent label connected with the solid support or first analyte-specific binding member,
an activator-labeled specific binding member including a second analyte-specific binding member and an activator connected with the second analyte-specific binding member;
a selective signal inhibiting agent, and
sample,
wherein the chemiluminescent-labeled immobilized specific binding member and activator-labeled specific binding member bind to analyte present in the sample to form a binding complex;
adding to the reaction mixture a trigger solution, wherein the trigger solution releases a detectable chemiluminescent signal correlated to the amount of the analyte-bound binding complex present in the reaction mixture.

2. The method of claim 1 wherein activator-labeled specific binding member comprises an activator connected to an analog of the analyte and wherein the analyte and the activator-labeled analog compete to bind with the chemiluminescent-labeled immobilized specific binding partner.

3. The method of claim 1 wherein the chemiluminescent-labeled specific binding member comprises a first analyte-specific binding member that is an analog of the analyte and wherein the analyte and the chemiluminescent-labeled specific binding member compete to bind with the activator-labeled immobilized specific binding member.

4. The assay method of claim 1 for an analyte in a sample, wherein forming a reaction mixture, in any order or concurrently, further includes enhancer.

5. The method of claim 1 wherein the selective signal inhibiting agent causes the ratio of signal produced by reaction between chemiluminescent label and activator in the binding complex with the analyte exceeds the signal from reaction between chemiluminescent label and activator when not in such a binding complex.

6. The method of claim 1 wherein the selective signal inhibiting agent is selected from the group consisting of aromatic compounds having at least two hydroxyl groups oriented in an ortho-, or para-relationship, aromatic compounds having at least a hydroxyl group and an amino group oriented in an ortho-, or para-relationship, compounds having at least two hydroxyl groups substituted on a C—C double bond, and nitrogen heterocyclic compounds.

7. The method of claim 1 wherein selective signal inhibiting agent is selected from the group consisting of ascorbate, isoascorbate, Trolox, L-Ascorbic acid 6-Palmitate, 5,6-Isopropylidene-L-Ascorbic acid, BHT, glutathione, uric acid, tocopherols, and catechin.

8. The method of claim 1 wherein the chemiluminescent-labeled immobilized specific binding member comprises a chemiluminescent label compound connected directly or indirectly to a specific binding member, wherein the chemiluminescent label is selected from aromatic cyclic diacylhydrazides, trihydroxyaromatic compounds, acridan ketenedithioacetal compounds, acridan esters, acridan thioesters, acridan sulfonamides, acridan enol derivatives, and a compound of the formula 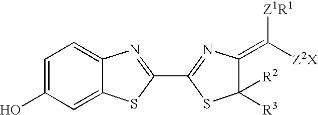
wherein R¹ is selected from alkyl, alkenyl, alkynyl, aryl, and aralkyl groups of 1-20 carbon atoms any of which can be substituted with 1-3 groups selected from carbonyl groups, carboxyl groups, tri(C₁-C₈ alkyl)silyl groups, a SO₃⁻ group, a OSO₃⁻² group, glycosyl groups, a PO₃⁻ group, a OPO₃⁻² group, halogen atoms, a hydroxyl group, a thiol group, amino groups, quaternary ammonium groups, or quaternary phosphonium groups, wherein X is selected from C₁-C₈ alkyl, aryl, aralkyl groups, alkyl or aryl carboxyl groups having from 1-20 carbon atoms, tri(C₁-C₈ alkyl)silyl groups, a SO₃⁻ group, glycosyl groups and phosphoryl groups of the formula PO(OR′)(OR″) wherein R′ and R″ are independently selected from C₁-C₈ alkyl, cyanoalkyl, aryl and aralkyl groups, trialkylsilyl groups, alkali metal cations, alkaline earth cations, ammonium and trialkylphosphonium cations, wherein Z¹ and Z² are each selected from O and S atoms and wherein R² and R³ are independently selected from hydrogen and C₁-C₈ alkyl.

9. The method of claim 1 wherein the chemiluminescent-labeled immobilized specific binding member comprises a chemiluminescent label compound connected directly or indirectly to a specific binding member, wherein the chemiluminescent label is a compound of the formula 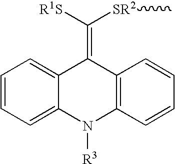
wherein [symbol] designates the point of attachment of the chemiluminescent label to the specific binding member, wherein R¹ and R² are independently selected from substituted or unsubstituted alkyl, substituted or unsubstituted alkenyl, substituted or unsubstituted alkynyl, substituted or unsubstituted aryl, and substituted or unsubstituted aralkyl groups of 1-20 carbon atoms, wherein when R¹ or R² is a substituted group, it can be substituted with 1-3 groups selected from carbonyl groups, carboxyl groups, tri(C₁-C₈ alkyl)silyl groups, a SO₃⁻ group, a OSO₃⁻² group, glycosyl groups, a PO₃⁻ group, a OPO₃⁻² group, halogen atoms, a hydroxyl group, a thiol group, amino groups, C(═O)NHNH₂, quaternary ammonium groups, and quaternary phosphonium groups, wherein R³ is selected from the group consisting of alkyl, substituted alkyl, substituted or unsubstituted alkenyl, substituted or unsubstituted alkynyl, substituted or unsubstituted aryl, substituted or unsubstituted aralkyl groups of 1-20 carbon atoms, phenyl, substituted or unsubstituted benzyl groups, alkoxyalkyl, carboxyalkyl and alkylsulfonic acid groups, wherein when R³ is a substituted group, it can be substituted with 1-3 groups selected from carbonyl groups, carboxyl groups, tri(C₁-C₈ alkyl)silyl groups, a SO₃⁻ group, a OSO₃⁻² group, glycosyl groups, a PO₃⁻ group, a OPO₃⁻² group, halogen atoms, a hydroxyl group, a thiol group, amino groups, C(═O)NHNH₂, quaternary ammonium groups, and quaternary phosphonium groups.

10. The method of claim 1 wherein the activator-labeled specific binding member comprises an activator compound connected directly or indirectly to a specific binding member, wherein the activator label is selected from transition metal salts, transition metal complexes and enzymes, wherein the activator label has peroxidase activity.

11. The method of claim 10 wherein the activator is a peroxidase enzyme.

12. The method of claim 1 wherein at least one of the chemiluminescent-labeled immobilized specific binding member and activator-labeled specific binding member comprises an auxiliary substance selected from soluble proteins, streptavidin, avidin, neutravidin, biotin, cationized BSA, fos, jun, soluble synthetic dendrimers, soluble synthetic polymers, soluble natural polymers, polysaccharides, dextran, oligonucleotides, liposomes, micelles, and vesicles.

13. The method of claim 4 wherein the enhancer is a compound or mixture of compounds that promotes the catalytic turnover of an activator having peroxidase activity.

14. The method of claim 13 wherein the enhancer is selected from phenol compounds, aromatic amines, benzoxazoles, hydroxybenzothiazoles, aryl boronic acids and mixtures thereof.

15. The method of claim 1 wherein the trigger solution comprises a peroxide compound.

16. The method of claim 1 wherein the trigger solution comprises an enhancer selected from phenol compounds, aromatic amines, benzoxazoles, hydroxybenzothiazoles, aryl boronic acids and mixtures thereof.

17. A kit for detecting an analyte in a sample comprising:
a first specific binding partner for the analyte;
a chemiluminescent compound conjugated to the first specific binding partner;
a second specific binding partner for the analyte; and
an activator compound conjugated to the second specific binding partner;
a solid support associated connected with either the chemiluminescent compound—first specific binding partner conjugate, or second specific binding partner-activator compound conjugate;
a selective signal inhibiting agent; and
a trigger solution.

18. The kit of claim 17 wherein the selective signal inhibiting agent is selected from the group consisting of aromatic compounds having at least two hydroxyl groups oriented in an ortho-, or para-relationship, aromatic compounds having at least a hydroxyl group and an amino group oriented in an ortho-, or para-relationship, compounds having at least two hydroxyl groups substituted on a C—C double bond, and nitrogen heterocyclic compounds.

19. The kit of claim 17 wherein the chemiluminescent compound is selected from aromatic cyclic diacylhydrazides, trihydroxyaromatic compounds, acridan ketenedithioacetal compounds, acridan esters, acridan thioesters, acridan sulfonamides, acridan enol derivatives, and a compound of the formula 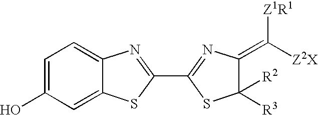
wherein R¹ is selected from alkyl, alkenyl, alkynyl, aryl, and aralkyl groups of 1-20 carbon atoms any of which can be substituted with 1-3 groups selected from carbonyl groups, carboxyl groups, tri(C₁-C₈ alkyl)silyl groups, a SO₃⁻ group, a OSO₃⁻² group, glycosyl groups, a PO₃⁻ group, a OPO₃⁻² group, halogen atoms, a hydroxyl group, a thiol group, amino groups, quaternary ammonium groups, or quaternary phosphonium groups, wherein X is selected from C₁-C₈ alkyl, aryl, aralkyl groups, alkyl or aryl carboxyl groups having from 1-20 carbon atoms, tri(C₁-C₈ alkyl)silyl groups, a SO₃⁻ group, glycosyl groups and phosphoryl groups of the formula PO(OR′)(OR″) wherein R′ and R″ are independently selected from C₁-C₈ alkyl, cyanoalkyl, aryl and aralkyl groups, trialkylsilyl groups, alkali metal cations, alkaline earth cations, ammonium and trialkylphosphonium cations, wherein Z¹ and Z² are each selected from O and S atoms and wherein R² and R³ are independently selected from hydrogen and C₁-C₈ alkyl.

20. The kit of claim 17 wherein the chemiluminescent-labeled immobilized specific binding member comprises a chemiluminescent label compound connected directly or indirectly to a specific binding member, wherein the chemiluminescent label is a compound of the formula 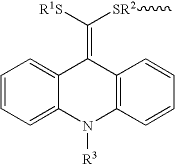
wherein [symbol] designates the point of attachment of the chemiluminescent label to the specific binding member, wherein R¹ and R² are independently selected from substituted or unsubstituted alkyl, substituted or unsubstituted alkenyl, substituted or unsubstituted alkynyl, substituted or unsubstituted aryl, and substituted or unsubstituted aralkyl groups of 1-20 carbon atoms, wherein when R¹ or R² is a substituted group, it can be substituted with 1-3 groups selected from carbonyl groups, carboxyl groups, tri(C₁-C₈ alkyl)silyl groups, a SO₃⁻ group, a OSO₃⁻² group, glycosyl groups, a PO₃⁻ group, a OPO₃⁻² group, halogen atoms, a hydroxyl group, a thiol group, amino groups, C(═O)NHNH₂, quaternary ammonium groups, and quaternary phosphonium groups, wherein R³ is selected from the group consisting of alkyl, substituted alkyl, substituted or unsubstituted alkenyl, substituted or unsubstituted alkynyl, substituted or unsubstituted aryl, substituted or unsubstituted aralkyl groups of 1-20 carbon atoms, phenyl, substituted or unsubstituted benzyl groups, alkoxyalkyl, carboxyalkyl and alkylsulfonic acid groups, wherein when R³ is a substituted group, it can be substituted with 1-3 groups selected from carbonyl groups, carboxyl groups, tri(C₁-C₈ alkyl)silyl groups, a SO₃⁻ group, a OSO₃⁻² group, glycosyl groups, a PO₃⁻ group, a OPO₃⁻² group, halogen atoms, a hydroxyl group, a thiol group, amino groups, C(═O)NHNH₂, quaternary ammonium groups, and quaternary phosphonium groups.

21. The kit of any claim 17 wherein the activator compound is selected from transition metal salts, transition metal complexes and enzymes, wherein the activator label has peroxidase activity.

22. The kit of claim 17 wherein the trigger solution comprises a peroxide selected from hydrogen peroxide, urea peroxide, and perborate salts.

23. The kit of claim 17 wherein the trigger solution comprises an enhancer selected from phenol compounds, aromatic amines, benzoxazoles, hydroxybenzothiazoles, aryl boronic acids and mixtures thereof.
